(19) United States
(12) Patent Application Publication
(10) Pub. No.: US 2022/0026441 A1
(43) Pub. Date: Jan. 27, 2022

(54) METHOD OF PROCESSING SAMPLES

(71) Applicant: Pataigin, LLC, Baltimore, MD (US)

(72) Inventors: Matthew Sorensen, Seattle, WA (US); Erik Nilsson, Seattle, WA (US)

(21) Appl. No.: 17/379,795

(22) Filed: Jul. 19, 2021

Related U.S. Application Data
(60) Provisional application No. 63/054,725, filed on Jul. 21, 2020.

(51) Int. Cl. G01N 33/68; G01N 21/65; G01N 33/92

(52) U.S. Cl. CPC G01N 33/6851; G01N 1/28; G01N 33/92; G01N 21/65

(57) ABSTRACT

Methods for processing samples are presented, said methods useful for, among other uses, collection and transport of liquid or other samples for analysis by mass spectrometry or other means, wherein a filament is used for collection or transport of samples. Methods for precisely positioning a filament and sensing material in contact with or adhering to a filament are presented. Said methods are in at least some aspects further useful for identification of microorganisms, bulk extraction of lipids, detecting infections and other diseases, and other purposes.

### BACKGROUND

### Technical Field

The present disclosure relates generally to the fields of analytical chemistry, microbiology, and medicine, and more particularly to diagnostic medicine, mass spectrometry, and microbial assays.

### Description of the Related Art

Various methods and systems exist for moving biological samples, such as in laboratories. Nevertheless, there remains room for improvement in such methods and systems, as well as in applying such methods and systems to new technical fields.

### BRIEF SUMMARY

A method may be summarized as comprising: using an apparatus to transfer material from a source location to a target location; and performing a spectroscopic analysis on the material at the target location; wherein the apparatus comprises a head portion configured to movably receive a filament having a first end for carrying the material, a transporting device configured to transport the head portion relative to the source location and relative to the target location, a driving device configured to advance the filament towards the head portion and to retract the filament away from the head portion, and a trimming device configured to trim the first end of the filament; wherein using the apparatus includes moving the first end of the filament into contact with the material at the source location, moving the first end of the filament and a portion of the material coupled to the first end of the filament from the source location to the target location, and moving the portion of the material coupled to the first end of the filament into contact with the target location to deposit the portion of the material at the target location; wherein using the apparatus includes, after the portion of the material is deposited at the target location, trimming the first end of the filament.

The filament may include a second end opposite the first end, wherein the second end of the filament is accommodated in a filament storage unit located at a fixed location relative to the head portion of the apparatus or at a fixed location relative to the driving device of the apparatus. The filament may include a second end opposite the first end, wherein the second end of the filament is accommodated in a movable filament storage unit. The driving device may be coupled to the head portion, mounted on a fixed support separated from the head portion, or mounted on a movable support separated from the head portion. The target location may not be in a well plate or receptacle.

A method may be summarized as comprising: transferring material from a source location to a target location by positioning a first end of a filament at the source location to collect material on the first end of the filament and then positioning the first end of the filament at the target location to deposit some or all of the material at the target location; and performing a spectroscopic analysis on the material at the target location.

The method may further comprise wiping, tamping, and/or vibrating the filament to encourage release of the material at the target location and/or to spread the material at the target location. The target location may be on a matrix-assisted laser desorption/ionization plate and the spectroscopic analysis may be matrix-assisted laser desorption/ionization mass spectrometry. The target location may be on a plate configured for use in Raman spectroscopy and the spectroscopic analysis may be Raman spectroscopy. The material may be derived from a biological sample and the material may be analyzed to determine the presence, concentration, or absence of one or more bacteria, fungi, viruses, protozoans, or other parasites or organisms. The material may be derived from urine, blood, a sample incubated in a blood bottle, sputum, endotracheal aspirate, bronchoalveolar lavage, feces, wound effluent, mucus, buccal swab, nasal swab, vaginal swab or secretion, nipple aspirate, sweat, saliva, semen or ejaculate, synovial fluid, cerebrospinal fluid, biopsy or other tissue sample, skin surface sample, tears, urinary catheter sample, culture plate, other clinical or medical sample, or another human, mammalian, or non-mammalian material. The sample may be a clinical sample and the spectroscopic analysis may be a diagnostic test.

Subsequent analysis may be performed to determine one or more of microbial species ID, microbial ID at a level of specificity above or below the level of species, antimicrobial resistance, antimicrobial susceptibility, microbial growth, and/or environmental response. The subsequent analysis may be performed entirely or predominantly on hydrophobic microbial molecules or lipids whether or not hydrophobic, including phospholipids. The subsequent analysis may include extracting microbial membrane lipids. The subsequent analysis may be for identifying and/or classifying microorganisms in one or more samples. The subsequent analysis may be for detecting and/or measuring antimicrobial resistance and/or susceptibility of a microorganism in one or more samples, and/or for estimating the minimum inhibitory concentration of a antimicrobial agent for a microorganism in one or more samples.

A method for preparing samples for MALDI or other mass spectrometric analysis may be summarized as comprising any combination of one or more processes described herein, in any order, using any modality. A method for performing MALDI or other mass spectrometric analysis may be summarized as comprising any combination of one or more processes described herein, in any order, using any modality. A method for extracting lipids, proteins, and/or similar molecules from samples may be summarized as comprising any combination of one or more processes described herein, in any order, using any modality. A method for identifying and/or classifying microorganisms in one or more samples may be summarized as comprising any combination of one or more processes described herein, in any order, using any modality. A method for detecting and/or measuring growth of microorganisms in a sample may be summarized as comprising any combination of one or more processes described herein, in any order, using any modality. A method for detecting and/or measuring one or more environmental responses and/or responses to a toxic or other substance of at least one microorganism or other organism, cell culture, or cell in one or more samples may be summarized as comprising any combination of one or more process described herein, in any order, using any modality. A method for detecting and/or measuring antimicrobial resistance and/or susceptibility of at least one microorganism in one or more samples, and/or for estimating the minimum inhibitory concentration of a antimicrobial agent for at least one microorganism in one or more samples may be summarized as comprising any combination of one or more processes described herein, in any order, using any modality. A method for treating infection, sepsis, or any other disease may make use of one or more methods and/or chemical compounds described herein, using any modality. A method for discovering, measuring, improving, and otherwise researching at least one environmental response of at least one organism singly or in combination with other species, strains, and/or phenotypes of organisms, may include any processes described herein, using any modality. A method for discovering, measuring, improving, and otherwise researching at least one substance with antimicrobial and/or other therapeutic properties may use any method described herein, using any modality.

### BRIEF DESCRIPTION OF THE SEVERAL VIEWS OF THE DRAWINGS

FIG. 1 shows an example of a plate.

FIG. 2 shows an apparatus configured for 3D printing with filaments.

FIG. 3 shows a microplate or a microtiter plate.

FIG. 4 shows a cell culture plate with culture media and microbial colonies.

FIG. 5A shows light transmitted by a filament, reflected, and subsequently detected.

FIG. 5B shows light transmitted in two directions by a filament and subsequently detected.

FIG. 6A shows light transmitted by a filament through an object and subsequently detected.

FIG. 6B shows light transmitted two directions by a filament and subsequently detected.

FIG. 7A shows light reflected, transmitted by a filament, and subsequently detected.

FIG. 7B shows light transmitted through a material, then transmitted by a filament and subsequently detected.

FIG. 8A shows a filament penetrating a surface.

FIG. 8B shows a filament submerged in a liquid.

FIG. 9A shows a filament in proximity to a non-orthogonal surface.

FIG. 9B shows a filament separated from a liquid.

FIG. 10A shows a filament with a mechanical transducer and a surface.

FIG. 10B shows a filament with a mechanical transducer and a liquid.

FIG. 11A shows a filament and a container.

FIG. 11B shows a filament and a liquid.

FIG. 12A shows a filament with a mechanical transducer and a container.

FIG. 12B shows a filament with a mechanical transducer submerged in a liquid.

FIG. 13A shows a filament with a mechanical transducer and a filament positioning device.

FIG. 13B shows a filament with a mechanical transducer, a filament positioning device, and a liquid.

FIGS. 14A-14C show example lipids for three classes of microbes.

FIG. 15 shows an example structure of lipid A.

FIG. 16 shows a filament cartridge mounted on a filament positioning device.

FIG. 17 shows a filament positioning device.

### DETAILED DESCRIPTION

The present disclosure comprises in at least one aspect one or more methods for manipulating one or more liquid and/or other samples in contact with an end of one or more filaments.

The present disclosure comprises in at least one aspect one or more methods for preparing samples for analysis by matrix-assisted laser desorption/ionization (MALDI) mass spectrometry or another mass spectrometry method in which a sample is prepared on and/or desorbed from a surface. The present disclosure comprises in at least one aspect one or more methods for preparing samples on a surface for analysis by a method other than mass spectrometry. For example, in at least one embodiment, at least one sample is prepared for analysis by Raman spectroscopy.

The present disclosure comprises in at least one aspect one or more methods for measuring the position of an end of a filament and/or positioning the end of a filament. For example, in at least one embodiment, an end of a filament is positioned so as to contact a surface of a liquid, and the contact of the filament end and surface of the liquid is detected by an optical or mechanical sensor, or by another method disclosed herein.

The present disclosure comprises in at least one aspect one or more methods for measuring the properties of a substance in contact with or in proximity to an end of a filament.

The present disclosure comprises in at least one aspect one or more methods for transferring and/or analyzing samples comprising volumes of less than 0.1 μL, between about 0.1 μL and 1 μL, about 1 μL, between about 1 μL and 10 μL, or greater than 10 μL.

The present disclosure comprises in at least one aspect one or more methods for analyzing two or more samples, in which carryover of signal between the two or more samples is to be minimized. For example, in at least one aspect, volumes from two or more bacterial liquid cultures are each transferred to at least one MALDI target location on a MALDI plate, and furthermore at least one of the at least two or more bacterial liquid cultures must not contaminate at least one other of the at least two bacterial liquid cultures.

The present disclosure comprises in at least one aspect one or more methods for preparing a sample for analysis by matrix-assisted laser desorption and ionization mass spectrometry (MALDI-MS or MALDI). In at least one embodiment at least one quantity of at least one liquid or other material with a volume of less than about 1 μL, about 1 μL, or more than about 1 μL is placed on a MALDI plate. In at least one embodiment, an apparatus incorporates a MALDI matrix sprayer, pipettor, or other applicator. In at least one embodiment, an apparatus incorporates a MALDI mass spectrometer or another kind of mass spectrometer.

In at least one embodiment, an apparatus incorporates a microscope, a Raman spectroscope, or another analytical instrument.

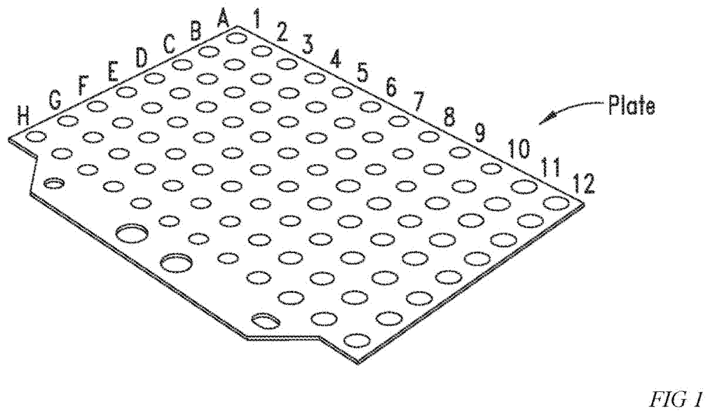

FIG. 1 illustrates a flat or substantially flat object of a type that may be used as a MALDI plate, commonly called a “plate.”

The flat object shown in FIG. 1 comprises one or more than one target location at which samples may be placed. The flat object in FIG. 1 has the locations at which samples may be placed marked or engraved on the surface. In at least one embodiment, the locations at which samples may be placed are or are not marked on the surface of a flat object.

In at least one embodiment, a plate or other substantially flat object is used to hold at least one sample for at least one kind of analysis such as without limitation surface enhanced Raman scattering (SERS), a type of Raman spectroscopy other than SERS, digital microfluidics, or another kind of analysis. In at least one embodiment, a plate or other substantially flat object is used to hold at least one sample for at least one type of microscopy. For example without limitation if the plate or other substantially flat object is made of a transparent material, the plate or other substantially flat object may be used to hold samples for transmission microscopy. Those skilled in the art will appreciate that for some types of analysis or types of sample, a plate or other substantially flat object may be highly flat and smooth, whereas for other types of analysis or sample types, a plate or other substantially flat object may not be flat or smooth or may not be highly flat or smooth. For example without limitation, the flatness and smoothness of microscope slides often affects optical performance of microscopy, whereas for MALDI mass spectroscopy, flatness is typically a lesser concern and smoothness a much lesser concern than with microscopy, while for desorption electrospray ionization (DESI) mass spectrometry, flatness and smoothness is usually of comparatively little importance.

In at least one embodiment, a plate or microfluidic device is used for digital microfluidics and/or another type of microfluidics. For example without limitation, in at least one embodiment, at least one quantity of at least one liquid and/or other substance is transferred from an end of a first filament onto a plate or microfluidic device, then optionally one or more microfluidic operations such as without limitation, mixing, dilution, concentration, moving droplets, evaporation, condensation, heating, cooling, and/or other processes and/or chemical reactions are performed on or with the at least one quantity of at least one liquid and/or other substance. Continuing with the example, optionally, an optical or other measurement or observation for example with a microscope or other optical device as described herein or familiar to those skilled in the art is made of the substance or a solution, residue, or other reaction product of the substance. Continuing with the example, optionally, the substance or a solution, residue, or other reaction product of the substance is collected onto an end of the first filament or an end of a second filament, wherein in at least one embodiment transfer of the substance or solution, residue, or other reaction product from the plate or microfluidic device to a filament is facilitated due to at least one of a chemical or physical property of the substance or a solution, residue, or other reaction product, transfer prior to collection by microfluidics or other means of the substance or solution, residue, or other reaction product to a location or region on the plate or microfluidic device with hydrophobicity higher than that of at least one of the first filament, the second filament, or at least one other region or location of the plate or microfluidic device, and/or the second filament having higher hydrophobicity less than at least one location or region of the plate or microfluidic device. In at least one embodiment, vibration and/or an electric field on one or more filaments and/or a plate or microfluidic device is used to effect or improve transfer of at least one quantity of at least one liquid and/or other substance from a first filament to the plate or microfluidic device and/or from the plate to the first filament or a second filament.

In at least one embodiment, at least one quantity of at least one liquid and/or other substance is placed on a plate or microfluidic device using at least one method described herein and at least one ambient mass spectrometric or similar technique is applied to the at least one quantity of at least one liquid and/or other substance, such as without limitation surface acoustic wave nebulization (SAWN) or DESI. In at least one embodiment, at least one microfluidic and at least one mass spectrometric operation are performed on at least one quantity of at least one liquid and/or other substance, using at least one method described herein. In at least one embodiment, at least one microfluidic operation and at least one measurement or other analytical operation including but not limited to mass spectrometry, spectroscopy, and microscopy are performed on at least one quantity of at least one liquid and/or other substance, using at least one method described herein or another method.

In at least one embodiment, a plate is a microfluidic device.

It will be appreciated by those skilled in the art that if a first material is more hydrophilic than a second material, the first material will have a hydrophobicity that is less than the hydrophobicity of the second material.

The present disclosure comprises in at least one aspect one or more methods for manipulating at least one elongated structure such as without limitation a tube, fiber, filament, wire, strand, thread, or capillary. Hereinafter except where context indicates otherwise, the aforementioned elongated structures are referred to collectively as “filaments.” In at least one embodiment a filament is a fiber in the sense of an optical fiber with or without cladding, a filament of a type used for 3D printing, a monofilament line of a type used for fishing, a thread of a type used for sutures in medicine and/or veterinary medicine, a thread of some other kind, a nylon string of a type use for weed trimmers, and/or any other suitable elongated structure. In at least one embodiment, at least one filament is composed of a plastic or polymer such as without limitation nylon, PMMA (polymethyl methacrylate), or polylactic acid (PLA). In at least one embodiment, at least one filament is composed of a metal, a mixture, a composite, or some other suitable material. In at least one embodiment, a filament is less than about 0.2 mm in diameter, between about 0.2 mm and 1.0 mm in diameter, about 1.0 mm in diameter, between about 1.0 mm and 5.0 mm in diameter, or more than about 5.0 mm in diameter. Those skilled in the art will readily appreciate what is meant by the diameter of a filament. On the end of a 1.0 mm filament, a liquid comprising water or predominantly water will typically form a drop with a volume of between about 0.5 μL and 1.0 μL. Similar volumes of non-liquid material will typically adhere to the end of a 1.0 mm filament. Within reasonable limits, to the end of a larger or smaller filament, a larger or smaller volume of material will adhere. In at least one embodiment, filament diameter is used to control the volume of material collected on the end of a filament. In at least one embodiment, filament diameter is used to control the volume of material transferred in one or more transfer operations. In at least one embodiment, a consistent filament diameter is used to control the variation in volume transferred and/or collected in two or more collection operations. In at least one embodiment, a consistent filament diameter is used to control the variation in volume transferred in two or more transfer operations.

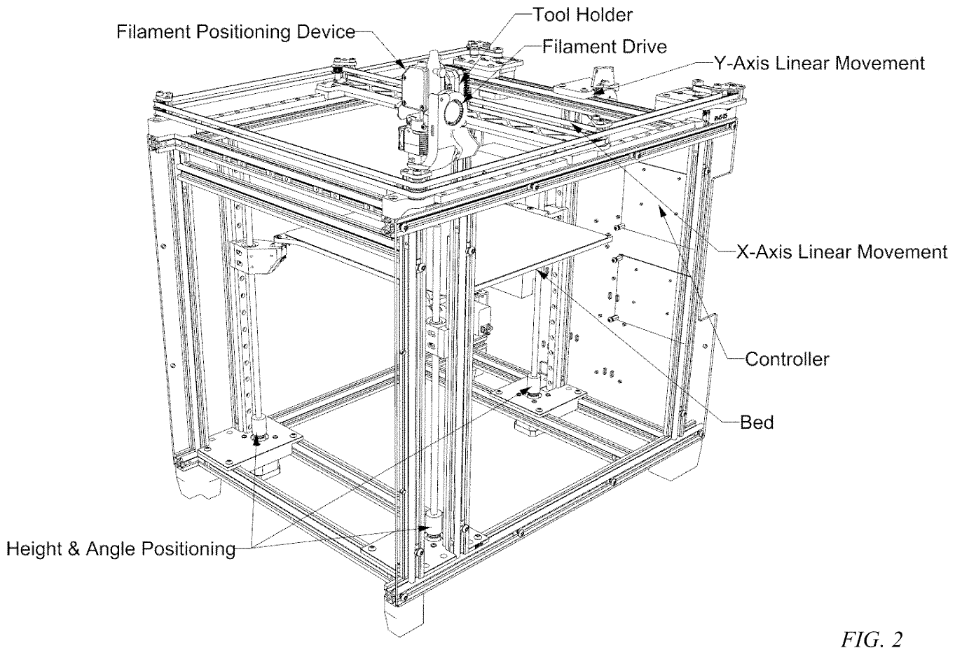

FIG. 2 illustrates an apparatus comprising in at least one aspect a motion platform, capable of automatic motion of at least one aspect of the aforementioned apparatus in at least one dimension and/or mechanical degree of freedom. Furthermore, the apparatus shown in FIG. 2 is or can be configured in at least one aspect to print 3D objects from filaments. A device configured for and/or used for 3D printing of objects is in many cases referred to by those skilled in the art as a “3D filament printer,” “filament printer,” or “3D printer.”

Motion platforms are used in a variety of automated apparatuses in addition to 3D printers, including without limitation laser cutters, milling machines, routers, apparatuses for working metal, wood, or other materials, apparatuses for liquid handling, apparatuses for automating chemical, biochemical, or biological processes, apparatuses for electronic part placement, apparatuses for imaging or inspection, and other apparatuses for factory or laboratory automation, or for manipulating or configuring objects robotically and/or under automatic control. Those skilled in the art will appreciate that a motion control platform as described herein may have any or all of the components and configurations typical of the aforementioned automated apparatuses and furthermore may have any or all of the components and configurations of the general class of motion platforms encompassing all such apparatuses described herein.

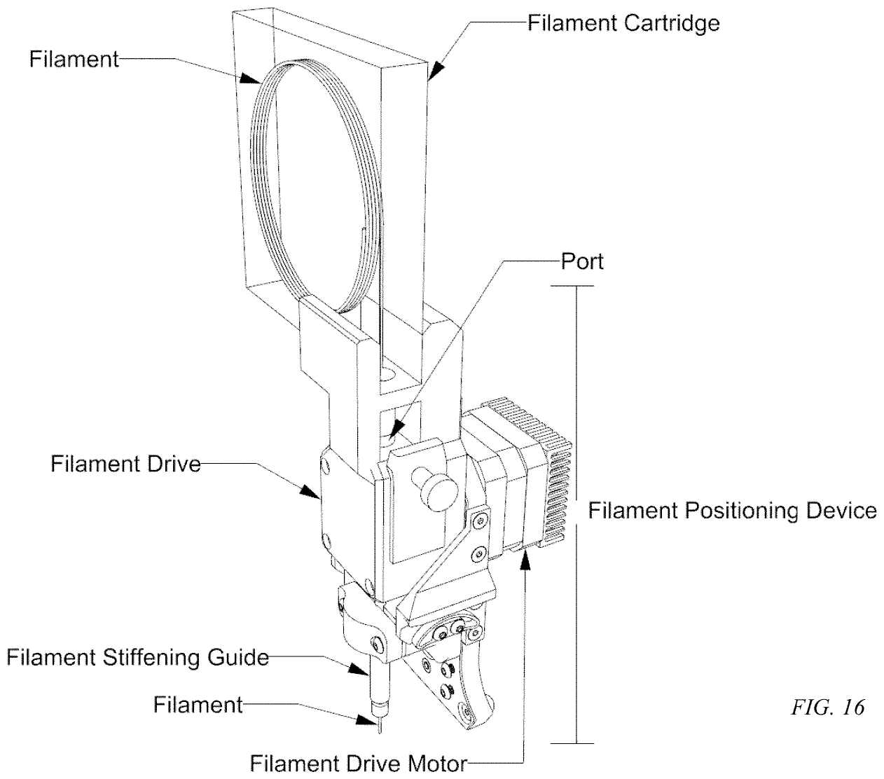

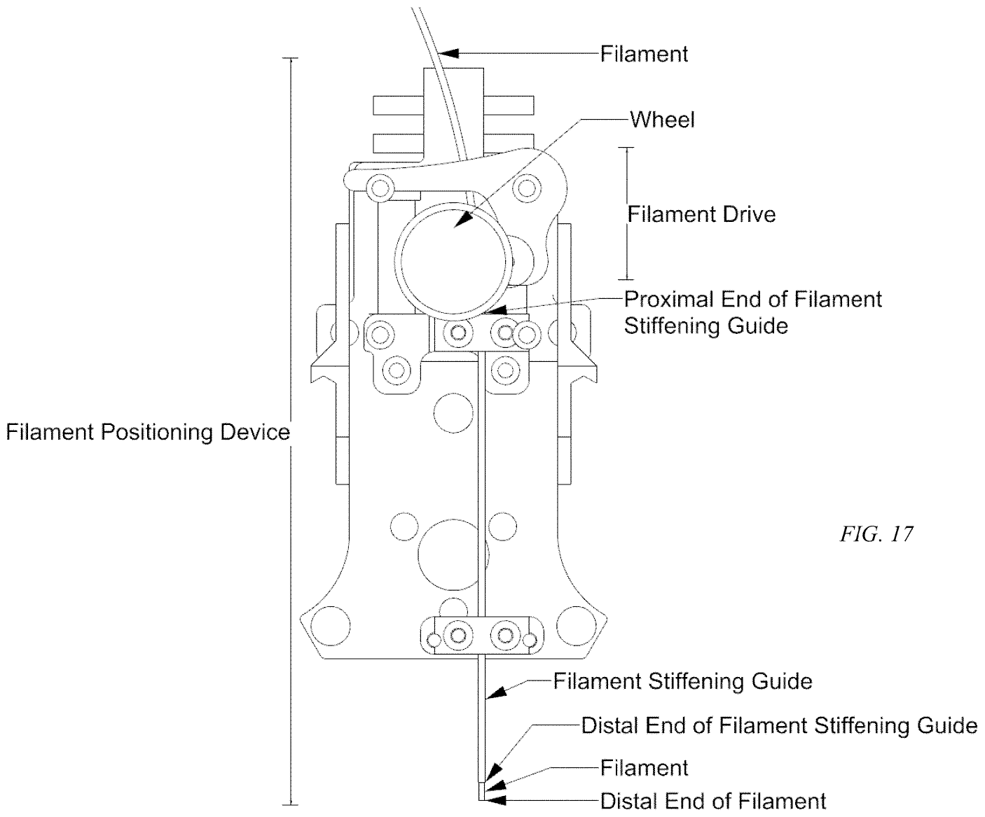

The present disclosure concerns in at least one aspect one or more methods for manipulating filaments using an apparatus, wherein the apparatus or at least one part of the apparatus is, resembles, or is configured as or similar to a motion platform and/or 3D filament printer for example without limitation the apparatus shown in FIG. 2. FIGS. 16 and 17 show an apparatus or a sub-assembly of an apparatus, said apparatus or sub-assembly comprising a sub-assembly of a motion platform substantially the same as the apparatus shown in FIG. 2. Such an apparatus may or may not include an extruder for 3D printing and/or a hot-end assembly for melting a filament. Such an apparatus may include a liquid dispenser comprising a liquid transporting tube, a needle connected to one end of the liquid transporting tube, and a supporting frame or similar apparatus for supporting and/or positioning the transporting tube and needle. Those skilled in the art sometimes refer to such a liquid dispenser as a “syringe dispenser” or “needle dispenser.” In at least one embodiment, a peristaltic pump, syringe pump or other pump is attached to the liquid transporting tube at the end that is not connected to the needle or at some other point on the tube, and liquid may be collected and/or dispensed via the needle. In at least one further embodiment, a liquid in a reservoir is dispensed through the transporting tube to the needle and subsequently dispensed by the needle. In at least one embodiment, a material is sprayed through a needle or similar apparatus to coat a surface with a liquid or for some other purpose. In at least one embodiment, a needle is not used, and liquid is collected and/or dispensed directly with the transporting tube. In at least one further embodiment, a positioning device feeds and/or withdraws a liquid transporting tube, and a contaminated region of a transporting tube can be cut off using any methods described herein for positioning a filament or any other method described herein. In at least one embodiment, an apparatus is used as described above, except a pipette tip or other nozzle, nebulizer, proboscis, nipple, sprayer, channel, orifice, or conduit replaces the needle in the apparatus. Such an apparatus may also include a tool for positioning a pen or similar marking device.

FIGS. 2, 16, and 17 show important aspects of distinctive features of motion platforms and filament printers. Returning now to FIG. 2, An X-linear movement and a Y linear movement are arranged in an XY movement so as to position a filament positioning device at horizontal XY position. The filament positioning device is commonly referred to as a “head.” In at least one embodiment, horizontal and/or vertical movement is not accomplished by two linear axes arranged as described above. For example without limitation, in at least one embodiment, a robotic arm is used.

The apparatus in FIG. 2 comprises multiple filament positioning devices and other devices such as cameras that can be mounted on the XY movement by means of a tool holder. The filament positioning devices of some filament printers can hold and position two or more filaments. One skilled in the art will appreciate that a tool holder and multiple heads allows positioning multiple filaments as well as positioning apparatuses that are not filaments, such as without limitation at least one camera. In at least one embodiment, an apparatus includes at least one camera that is in a stationary position or is otherwise not on an XY movement. Those skilled in the art will appreciate that in at least one embodiment, an apparatus contains a motion platform and also contains one or more cameras to inspect the results of operations, locate fiducials or other objects, measure a size, volume, opacity, fluorescence, concentration, turbidity, birefringence, and/or other property of an object, measure a distance between at least two objects or an orientation or other relationship between at least two objects, or for other purposes related to the purpose of the apparatus. In at least one embodiment, at least one camera is used to inspect results of 3D printing. In at least one embodiment, at least one camera is used to locate a MALDI plate, microtiter plate, culture plate, or other container or other object, to locate a substance or sample of material within, in, or on a MALDI plate, microtiter plate, culture plate, or other container or other object, or to determine the height, slope, or surface map of a MALDI plate, microtiter plate, culture plate, or other container or other object. In at least one embodiment, at least one camera is used to measure the size or estimate the composition, quality, orientation, or other characteristics of an object. In at least one embodiment, at least one camera is used to estimate the height of the surface of culture media in a culture plate.

Those skilled in the art will appreciate that by “camera” is meant a variety of optical devices for capturing images and/or measuring photonic emissions and/or spectra, which may be combined with or include a variety of optical components suited for these purposes. For example without limitation, a camera combined with a microscope or optical elements having the effect of magnifying an image comprises a camera herein, unless context indicates otherwise.

Continuing now with the description of FIG. 2, tool docks are provided for the stowage of tools while they are not mounted on the tool holder. In at least one embodiment, an apparatus includes tool docks. In at least one embodiment, an apparatus does not include tool docks. In at least one embodiment, more than one tool may be mounted on a tool holder, by means of having more than one mount position on the tool holder, and/or by means of at least one tool that may itself have mounted on it a second tool.

In at least one embodiment, an apparatus contains one or multiple filament positioning devices. In at least one embodiment, an apparatus contains a filament positioning device that can position one filament. In at least one embodiment, an apparatus contains a filament positioning device that can position multiple filaments.

In one embodiment, at least one filament positioning device contains a filament drive that positions a filament vertically, holds the vertical position of a filament, and draws filament from a storage location optionally through a guide to the filament positioning device. For example, without limitation, a filament drive can comprise two wheels mounted with their rims facing each other, with the filament passing between the wheels, and one or both wheels driven by a motor to raise or lower the end of the filament. A filament positioning device is optionally provided with a guide comprising a tube or similar component. A filament passes from a filament storage area through the guide to a filament positioning device. In at least one embodiment, a filament leaves a filament storage area before entering a guide and leaves a guide before entering a filament positioning device. In one embodiment, a guide is composed of a material with a low coefficient of friction such as without limitation polytetrafluoroethylene (PTFE, commercially available under the brand name TEFLON) to reduce the friction of filament travelling through the guide. In at least one embodiment, a storage area for a filament is on a filament positioning device. In at least one further embodiment, an apparatus does not contain a guide between a filament positioning device and a filament storage area.

One skilled in the art will appreciate that with respect to filament positioning, “vertically” is meant respective to the elongated dimension of a filament, which may not correspond to an actual vertical direction, and furthermore, “vertically” can apply in an approximate sense, depending on the design of the filament positioning device.

One skilled in the art will appreciate that with respect to filament positioning, a filament positioning device may be moved in a single axis, herein referred to as X, movement in two axes and/or in a plane, herein referred to as XY, or movement in 3-dimensional space, herein referred to as XYZ. One skilled in the art will further appreciate that movement in a polar coordinate space or another space with polar, linear, and/or other degrees of freedom can be expressed as movement in X, XY, or XYZ. For the sake of simplifying discussion, movement of an object in an X, XY, or XYZ direction is understood to describe movement and/or configuration of the object in space with any degrees of freedom and/or any geometric constraints on position or motion.

An apparatus in which a filament drive is located on or with a filament positioning device has the advantage of positioning a filament more precisely than an apparatus in which a filament drive is placed at another location, for example in an apparatus a drive is located between a guide and a filament storage area. If the filament drive is not located on the filament positioning device, as the filament positioning device moves to different X, XY, or XYZ positions, the guide flexes and the filament will extend and retract in the guide, changing vertical position in the filament positioning device. However, in at least one embodiment, an apparatus is used with a filament drive that is not located in a filament positioning device. In at least one further embodiment, an apparatus is used with a guide in between a filament drive and a filament positioning device, as is typical for many filament printer designs. In at least one further embodiment, the aforementioned vertical motion of a filament is measured using optical or mechanical means described herein and then compensated for, or equivalently the distance from the end of a filament end at a particular X or XY location to a surface or interface can be detected or measured or the proximity of a filament end to a surface or interface can be detected or measured, and thereby the aforementioned disadvantages of vertical motion due to guide flexing can be reduced by detecting the position or proximity of a filament end. In at least one embodiment, the relative height of an end of a filament in a filament positioning device at different X, XY, or XYZ locations is estimated from said X, XY, or XYZ locations and prior measurements and/or an estimate of effective length of the guide at said X, XY, or XYZ position, and the height of the filament end is adjusted with a filament drive or the estimated height of the filament end is compensated for in subsequent operations.

Continuing now with the description of FIG. 2, a bed can be automatically raised and lowered, allowing the distance from a holder and/or from a filament positioning device to a bed to be precisely controlled. A means of levelling a bed is typically provided. FIG. 2 shows three independent lead screws controlling the height of three points on the perimeter of the bed, allowing both the height and angle of the bed to be precisely controlled. The bed is sometimes referred to as a “baseplate” or “base.” As an alternative or in addition to a means for leveling and/or controlling the height and/or angle of a bed, in at least one embodiment, an apparatus has a means of optically or mechanically measuring the height and/or angle of a bed, and compensating in subsequent motion of elements of the apparatus for the aforementioned height and/or angle of the bed. It will be appreciated by those skilled in the art that by “angle” of a bed is meant a slope and direction of slope, equivalently two angles measured on non-parallel axes, and/or a geometric description of the location in space of a planar or non-planar surface.

In at least one embodiment, an apparatus has more than one bed, or has at least one stage movable in at least one of the X, Y, and Z direction and/or one or more rotation directions that is mounted on a bed. In at least one embodiment, in addition or alternative to a bed that can be raised and lowered, the filament positioning device can also be raised and lowered by similar means, by raising the tool holder if any, by raising the filament position device itself, or by any appropriate means having the same effect. In at least one embodiment, in addition or alternative to a bed and/or filament positioning device that can be raised and lowered, the filament positioning device raises and lowers a filament, changing the height of the filament end relative to at least one bed or stage.

In at least one embodiment an apparatus is used in which a bed can be heated and/or cooled. Those skilled in the art will appreciate that a heated bed is a typical feature of filament printers, incubators, and other apparatuses. In at least one embodiment, an apparatus is used in which a bed has separate zones that can be heated and/or cooled to different temperatures. In at least one embodiment, the temperature of a bed is measured at one or more locations, and the aforementioned temperature measurements are used to control or adjust the temperature or temperatures of the bed, adjust some other process parameter relative to the temperature measurements, determine if the temperature of the bed is at a specific value or within a specific range of values, or some other suitable purpose related to the function of the apparatus.

In at least one embodiment, a chamber is provided for heating or incubating at least one sample. In at least one embodiment, at least one chamber is provided for heating or incubating at least one sample on at least one MALDI plate, microscope slide, or other substantially flat or planar surface. In at least one embodiment, at least one chamber is provided for heating or incubating at least one sample in a well of at least one microtiter plate, culture plate, or other container. In at least one embodiment, an apparatus is used to move at least one MALDI plate, microscope slide, or other substantially flat or planar surface or microtiter plate, culture plate, or other container into and/or out of at least one chamber, and/or for moving a lid or cover. For example without limitation, in at least one embodiment, an apparatus includes gripping tool capable of gripping a MALDI plate that can be mounted on a tool holder. In at least one embodiment, a chamber is formed by placing a cover or lid on a location of a bed. In at least one embodiment, incubation and/or heating is accomplished by means of a heated bed without a separate cover or chamber. Those skilled in the art will appreciate that by “incubation” is meant any process in which a substance is modified by a process that comprises in at least one aspect heating the substance and/or controlling the temperature, temperature range, or temperature profile or sequence of the substance.

In at least one embodiment a filament cutter that cuts off contaminated or otherwise undesirable sections of filament is mounted on a bed, a filament positioning device, or a part of the apparatus other than a bed or a filament positioning device. In at least one embodiment, a receptacle for filament sections is positioned relative to a filament cutter such that filament sections cut by the filament cutter are deposited in a filament receptacle.

In at least one embodiment, an apparatus is used wherein a filament positioning device includes a filament stiffening guide. Filament passes through or along the filament stiffening guide, which allows the filament to be positioned with greater accuracy and/or allows the filament to press or penetrate objects with greater force than an equivalent apparatus without a filament stiffening guide.

Continuing now with the description of FIG. 2, a controller is connected to the appropriate elements of the apparatus and controls and coordinates their function, sending commands and receiving inputs from sensors.

Filament printers typically melt filament and apply it as a liquid. The present disclosure comprises in at least one aspect one or more methods in which filaments are not melted. The present disclosure comprises in at least one aspect one or more methods in which at least one filament is melted and/or manipulated by cutting, melting, abrasion, stretching, compression, polishing, curing, bending, or modification by heat or light. The present disclosure comprises in at least one aspect one or more methods in which fixturing, tooling, containers, covers, alignment marks, guides, and/or other components of an apparatus as described herein are 3D printed by an apparatus as described herein on a bed and/or on a MALDI plate, microtiter plate, culture plate, stage, or other fixture or object. For example without limitation, in at least one embodiment, outlines or pockets or similar features are 3D printed to indicate placement, retain in position, and/or align an object in the apparatus. For example without limitation, in at least one embodiment outlines for culture plates and MALDI plates are printed on a bed, the bed with outlines or pockets subsequently useful for transferring microbial colonies from culture plates to MALDI plates. In at least one other embodiment, outlines or pockets or similar features for placement of for example culture plates and MALDI plates are printed or etched in a bed using a temporary or permanent process other than 3D printing. In at least one embodiment, an apparatus comprises in part a removable cover that is printed, marked, or etched instead of the bed itself. In at least one embodiment, an apparatus contains a bed that is removable. In at least one embodiment, a non-permanent marking device marks outlines for placement of objects on at least one bed or cover; for example a tool holder holds a pen marking tool that is used to mark a bed.

The present disclosure comprises in at least one aspect one or more methods for determining the position of an end of a filament by means of light carried by at least one optical mode of the filament, of the filament and any cladding, of the filament and the surrounding air or other atmosphere and/or solution, of the filament and the air or other atmosphere and/or solution in an interior bore of the filament, or of any combination of these. The present disclosure comprises in at least one aspect one or more methods for determining the position of a filament by means of changes in the amount of light transmitted into, out of, or into and out of the end of a filament in the vicinity of an air/liquid boundary, a liquid/liquid boundary, an air/solid boundary, a liquid/solid boundary, or any similar boundary.

The present disclosure comprises in at least one aspect one or more methods for determining the position of an end of a filament by means of change in at least one of resistance, vibrational mode, or other mechanical properties caused by contact or proximity of the filament with a liquid, a solid, or a substance other than a liquid or a solid.

In at least one embodiment, precisely determining the position of an end of a filament allows reliable collection of microbial cells from liquid and/or solid culture media.

In at least one further embodiment, at least one of the following quantities is known with relatively low precision regarding one or more sampling locations and/or has significant variation for two or more sampling locations: a top surface location, a bottom location, the smoothness of a surface, or the location of an enclosing surface; wherein the sampling location is a liquid, a semisolid such as culture media, a solid, or some other material; wherein the sampling location is enclosed as in a microwell plate, culture plate, or microplate, not enclosed as in a drop on a surface, or in any other suitable configuration. In at least one further embodiment to the aforementioned further embodiment, positioning of a filament relative to a boundary allows more precise, rapid, and/or reliable material collection and/or transfer. In at least one further embodiment to the aforementioned further embodiment, positioning of a filament relative to a boundary prevents proximity or collision of two or more elements of an apparatus and/or reduces the force and/or energy with which two or more elements of an apparatus contact, thereby preventing damage, spillage, contamination, carryover, wastage, and/or some other undesired effect. For example without limitation, in at least one embodiment, a filament is positioned so as to contact the top surface of a liquid volume so as to collect a drop of liquid without splashing the liquid.

The present disclosure comprises in at least one aspect one or more methods for measuring a physical property of a sample in contact with or in proximity of a filament by means of light carried by at least one optical mode of the filament, of the filament and any cladding, of the filament and the surrounding air or other atmosphere and/or solution, of the filament and the air or other atmosphere and/or solution in an interior bore of the filament, or of any combination of these. The present disclosure comprises in at least one aspect one or more methods for measuring a physical property of a sample by means of changes in the amount of light transmitted into, out of, or into and out of the end of a filament at or in the vicinity of an air/liquid boundary, a liquid/liquid boundary, an air/solid boundary, a liquid/solid boundary, or any similar boundary.

The present disclosure comprises in at least one aspect one or more methods for measuring a physical property of a sample by means of a change in at least one of resistance, vibrational mode, or other mechanical property caused by contact of the filament with a liquid, a solid, or a substance other than a liquid or a solid.

For example without limitation, in at least one embodiment, the fluorescence of a sample is measured by collecting a sample onto the end of a filament, illuminating the sample with light by illuminating the filament, and detecting fluorescence by detecting fluorescence illuminating the filament from the sample. In a further example, in at least one embodiment, the fluorescence of a sample on the end of a filament is measured, and the sample on the end of a filament is not illuminated by the filament or fluorescence is detected by a method not involving the filament.

The present disclosure comprises in at least one aspect one or more methods for identifying or measuring the presence or quantity in a sample of one or more microbial species, one or more microbial taxa above the level of species, and/or one or more microbial strains.

The present disclosure comprises in at least one aspect one or more methods for identifying or measuring in a sample, for one or more microbial species, one or more microbial taxa above the level of species, and/or one or more microbial strains, antimicrobial susceptibility or resistance, virulence, and/or one or more other categories such as without limitation Gram stain.

The present disclosure comprises in at least one aspect one or more methods for estimating the quantity of one or more microbial species, one or more microbial taxa of species, and/or one or more strains in a sample.

The present disclosure comprises in at least one aspect one or more methods for diagnosing a microbial infection or other disease, and/or making estimates or predictions about the past, present, or future status of a disease.

The present disclosure comprises in at least one aspect one or more methods for extracting lipids and/or other molecules from samples.

Infectious diseases remain a serious health burden throughout the world. Diagnostic tests are critical to treating infectious diseases, but current tests are slow and lack sensitivity. Faster, more sensitive, and/or more accurate tests would allow clinicians to give the right treatment to patients sooner. Protein fingerprinting tests such as MALDI Biotyper and Vitek-MS have shown that matrix-assisted laser desorption and ionization (MALDI) mass spectrometry can be used for a cost-effective, multiplex microbial test. Herein, a “multiplex” test is a test that simultaneously tests for the individual presence of multiple microbial species, strains, taxa, and/or other phenotypes such as antimicrobial resistance. At least one embodiment of the present disclosure comprises a method for preparing samples for MALDI analysis and for identifying and/or measuring microbes from such samples.

The present disclosure comprises in at least one aspect one or more methods for transferring microbial cells and/or other substances from a solid medium to one or more other solid mediums and/or one or more liquid solutions and/or one or more flat surfaces.

The present disclosure comprises in at least one aspect one or more methods for transferring microbial cells and/or other substances from a liquid solution to one or more other liquid solutions and/or one or more solid mediums and/or one or more flat surfaces.

The present disclosure comprises in at least one aspect one or more methods for transferring microbial cells and/or other substances from a flat surface to one or more other flat surfaces and/or one or more liquid solutions and/or one or more solid mediums.

Herein, unless context indicates otherwise, a liquid or a liquid solution can be an emulsion, suspension, colloid, slurry, semiliquid, semisolid, or other substance having properties in common with a liquid such that there is at least one method described herein that is applicable to a liquid and said at least one method can be applied to the other sub stance.

Herein, unless specified otherwise or clear from context, “sensitivity” means the ability to identify a microbe from a small number of organisms in a sample. That is, high sensitivity implies a low limit of detection (“LOD”).

The present disclosure relates to methods for processing one or more samples and/or components of samples, to ascertain or estimate facts about said samples, and/or to extract specific chemicals or classes of chemicals from said samples. In at least one embodiment, at least one sample is a biological sample. In at least one embodiment, said biological sample contains, may contain, and/or is suspected to contain at least one microbial species. In at least one embodiment, said a least one microbial species is one or more of bacteria, archaea, fungi, or protozoa.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof.

“Microbial organism,” “microbe,” or “microorganism” as used herein interchangeably refers to any one of a bacterial species; an archaea; a yeast and/or fungal species; or any microbial species other than bacteria, yeast, or fungi, e.g. protozoa. A microbial organism may exist as a single cell or in a colony of cells.

As used herein, “antimicrobial,” or “treatment” are used interchangeably to mean any agent used to kill (microbicidal) or retard the growth of (biostatic) a microorganism. Antimicrobial medicines include, but are not limited to, antibiotics and antifungals.

Lipid A, the endotoxic portion of lipopolysaccharide (LPS) is embedded in the outer leaflet of the Gram-negative bacterial outer membrane. As an essential component of Gram-negative bacterial membranes, lipid A exhibits species-specific structural diversity. The general structure consists of a backbone of two glucosamine residues present as a B-(1-6)-linked dimer. This backbone can be diversified in response to specific environmental signals or between bacterial species. Specifically, changes in the fatty acid content varying both in the length and number of fatty acid side chains (e.g. tetra- to hepta-acylated) and phosphorylation patterns can differ as well. Additional modifications of the phosphate residues by monosaccharides, such as aminoarabinose or galactosamine and phosphoethanolamine can occur. The diversity of such species and environmentally-driven structural modifications are an adaptive mechanism that increases bacterial survival often through increasing resistance to hostantimicrobial peptides, or in the avoidance of the host innate immune system. Precursor molecules (i.e.: molecules from which LA is cleaved during isolation) to LA include, but are not limited to LPS.

Lipoteichoic acid (LTA) is a major cell wall component of Gram-positive bacteria.

The Gram-positive cell wall is composed of cross-linked peptidoglycan (PG) variably decorated with teichoic acid polymers. Teichoic acid polymers are also linked to plasma membrane phospholipids. The general structure of LTA varies between species consisting of 2 or 4 acyl groups, of variable chain length. LTA from low G+C subdivisions of Gram-positive bacteria contains two fatty acid tails, while those from high G+C bacteria contain 4 fatty acid tails. Additionally, LTA can be variably modified with alanine (in response to low pH), or glycosyl linkages depending on bacterial background. Glycosyl linkages can include glycerol phosphate, galactose, or N-acetyl-glycerol.

In at least one embodiment, a sample is processed, said sample being one or more of a culture plate colony or smear, a broth culture sample, a blood culture sample, a sample from a biofluid, a clinical sample, or a nonclinical sample. In at least one embodiment, a sample is one or more of an environmental sample, a veterinary sample, an agricultural sample, a food or food safety sample, an industrial sample, a process control sample, or a forensic sample. In at least one embodiment, the aforementioned biofluid is a biofluid from a human or non-human source. In at least one embodiment, a sample is processed, said sample comprising, derived from, or obtained from a urine specimen, a blood sample, a sample incubated in a blood bottle, sputum, endotracheal aspirate, bronchoalveolar lavage, feces, wound effluent, mucus, buccal swab, nasal swab, vaginal swab or secretion, nipple aspirate, sweat, saliva, semen or ejaculate, synovial fluid, cerebrospinal fluid, biopsy or other tissue sample, skin surface sample, tears, a urinary catheter sample, a culture plate, or another clinical or medical sample, or another human, mammalian, or non-mammalian material. In at least one embodiment, a sample comprises a substance or assembly containing at least one component that is a sample as described herein.

A sample can be used as obtained, or can be processed in any way suitable for use with the methods of this disclosure. In one embodiment, the methods comprise identifying bacteria directly from a complex sample (i.e., no requirement for amplifying bacteria present in the sample). In another embodiment, bacteria are isolated from the sample, such as by streaking onto solid bacterial culture medium, followed by growth for an appropriate period of time.

The following embodiments and description describe embodiments and aspects of the disclosure, without limiting the disclosure in any way. Specific embodiments are numbered and referred to as “EMBODIMENT 101,” etc. Also, certain embodiments are provided by way of example for one or more embodiments or the like; such descriptions are not referred to by number.

FIG. 1 illustrates a substantially flat object, such substantially flat object hereinafter referred, except when context indicates otherwise, as a “MALDI plate” or “plate.” The plate in FIG. 1 has at least one region called a “spot” at which in at least one embodiment, at least one material is placed. In FIG. 1, spots of the plate are indicated by circles, as is typical, but in at least one embodiment, at least one spot is indicated by a mark other than a circle or is not indicated by any visual mark or structure.

Some parts of the description describe operations involving a single sample and/or a single spot. One skilled in the art will appreciate that a plate may contain more than one spot such as without limiting the disclosure 96 spots or 384 spots. Thus, one skilled in the art will appreciate that, in at least one embodiment, multiple samples are applied to one or more spots on a plate. Furthermore, in at least one embodiment, a single sample is applied to more than one spot. Thus, in at least one embodiment, in at least one sequence of steps, at least one such step is applied once or multiple times to one or more samples and/or one or more spots, whereas at least one other such step is applied to a plate as a whole or to a region of a plate as a whole, so said one other step is not specifically applied per sample or per spot. One skilled in the art will appreciate that different embodiments of the present disclosure may be practiced using the same or different spots of one or more plates, while optionally the said one or more plates may have spots which are not used or which are used but do not constitute the practice of any embodiment herein. For example without limitation, a sample can be placed on two or more spots, and at least one spot of said two or more spots is used to extract lipids according to a method comprising an embodiment of the present disclosure, while before, after, or in parallel, at least one other spot of said two or more spots is used for some other purpose. Likewise, one embodiment can be practiced using at least one spot on a plate, and a different embodiment can be practiced using at least one other spot on the plate.

In at least one embodiment, a plate is used that is made out of steel such as stainless steel. In at least one embodiment, a plate is used that is made out of a metal other than steel. In at least one embodiment, a plate is used that is made out of a material that is not a metal. In some embodiments the stainless steel has been treated by one or more of passivating, pickling, and electropolishing.

In at least one embodiment, a plate is used that is a composite structure, for example without limiting the disclosure, having a first layer of one material, and a second layer of a different material situated on top of the first layer, such that the second layer has different chemical properties affecting the shape, composition, and/or movement of liquids on said surface. In at least one embodiment, the aforementioned second layer covers only part of the first layer. For example without limiting the disclosure, in at least one embodiment, a plate is used that comprises a first layer made of stainless steel and a second layer of a hydrophobic material, wherein the hydrophobic material does not cover some or all of one or more spots on the plate and instead covers some or all of the surface of the plate other than said one or more spots. As a second example without limiting the disclosure, in at least one embodiment, a plate is used that comprises a first layer made of stainless steel and a second layer of a lipophilic material, wherein the lipophilic material covers some or all of at least one spot. As a third example without limiting the disclosure, in at least one embodiment, a plate is used that comprises a first layer made of stainless steel and a second and a third layer said second and third layers having at least one difference in their chemical properties, and said second and third layers may or may not overlap.

In at least one embodiment, at least one surface of the plate has been inscribed, etched, or otherwise modified with a pattern of raised or lowered markings or structures, or a pattern of both raised and lowered markings or structures, such markings or structures provided for identifying visual locations to an operator and/or affecting the composition, shape, and/or improving and/or retarding movement of liquids on said surface. In at least one embodiment, at least one surface of the plate does not have such aforementioned markings or structures.

In at least one embodiment, one or more layers of a plate are passivated or otherwise chemically treated or modified. For example without limiting the disclosure, in at least one embodiment a stainless steel plate has been treated with an acid such as citric or nitric acid to passivate the plate.

In at least one embodiment, a substance is placed on a spot and rests on or adheres to the spot, so that the spot can be said to “hold” the substance, or also the spot can be said to “contain” the substance, even though the spot may or may not comprise a container in the ordinary sense. Furthermore, the spot can be said to contain the substance, even if there is no definite boundary of the spot, or even if there is at least one definite boundary of the spot but said substance plated on the spot is only partially within said at least one definite boundary.

In at least one embodiment, at least one chemical reagent or other material is applied to at least one spot. For example without limiting the disclosure, in at least one embodiment, a mixture of citric acid and sodium citrate is applied to at least one spot. As a further example without limiting the disclosure, in at least one embodiment, sodium acetate is applied to at least one spot. As a further example without limiting the disclosure, in at least one embodiment, a material that acts as a MALDI matrix is applied to at least one spot. As a further example without limiting the disclosure, in at least one embodiment, more than one material is applied to at least one spot. In some embodiments, the MALDI matrix is between about 0.5 μL and 2 μL or about 1 μL of a solution comprising an about 12:6:1 ratio mixture of chloroform:methanol:water to which about 10 mg/mL of beta-carboline has been added. In at least one embodiment, different combinations of materials are applied to two or more different spots. In at least one embodiment, materials are applied to at least one spot but not to at least one other spot. In at least one embodiment, at least one chemical reagent or other material is applied as part of manufacturing the plate. In at least one embodiment, at least one chemical reagent or other material is applied after the plate is manufactured. In at least one embodiment, a kit comprises a plate and one or more reagents, said one or more reagents to be placed on one or more spots by the user of the kit.

In various non-limiting embodiments, the methods in the present disclosure can be used to identify one or more bacteria (or sub-species thereof) including but not limited to Escherichia coli, Staphylococcus aureus, Staphylococcus epidermidis, Streptococcus pneumoniae, S. mitis, Streptococcus pyogenes, Stenotrophomonas maltophila, Mycobacterium tuberculosis, Neisseria gonorrhoeae, Neisseria meningitidis, Bordetella pertussis, B. bronchioseptica, Enterococcus faecalis, Salmonella typhimurium, Salmonella choleraesuis, Klebsiella pneumoniae, Pseudomonas aeruginosa, Acinetobacter baumannii, A. calcoaceticus, Bacteroides nordii, B. Salversiae, Enterobacter Subspecies including E. asburiae, E. cloacae, E. hormaechei, E. kobei, E. ludwigii, and E. nimipressuralis, extended spectrum B-lactamase organisms, as well as bacterium in the genus Acinetobacter; Actinomyces, Bacillus, Bacteroides, Bordetella, Borrelia, Brucella, Clostridium, Corynebacterium, Campylobacter, Deinococcus, Escherichia, Enterobacter, Enterococcus, Erwinia, Eubacterium, Flavobacterium, Francisella, Gluconobacter, Helicobacter; Intrasporangium, Janthinobacterium, Klebsiella, Kingella, Legionella, Leptospira, Mycobacterium, Moraxella, Neisseria, Oscillospira, Proteus, Pseudomonas, Providencia, Rickettsia, Salmonella, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

In some of the following embodiments, a single sample and/or a single spot is used. In at least one embodiment, a plate has multiple spots. In at least one embodiment, steps below applied to a single spot are performed two or more times to two or more spots of the same plate and/or of different plates, with the same or different samples or specimens used with different spots. In some of the following embodiments, steps applied to a plate as a whole have an effect on some or all of the spots of said plate. The various embodiments described herein can be combined to provide further embodiments.

In at least one embodiment an apparatus is used to collect or transfer material.

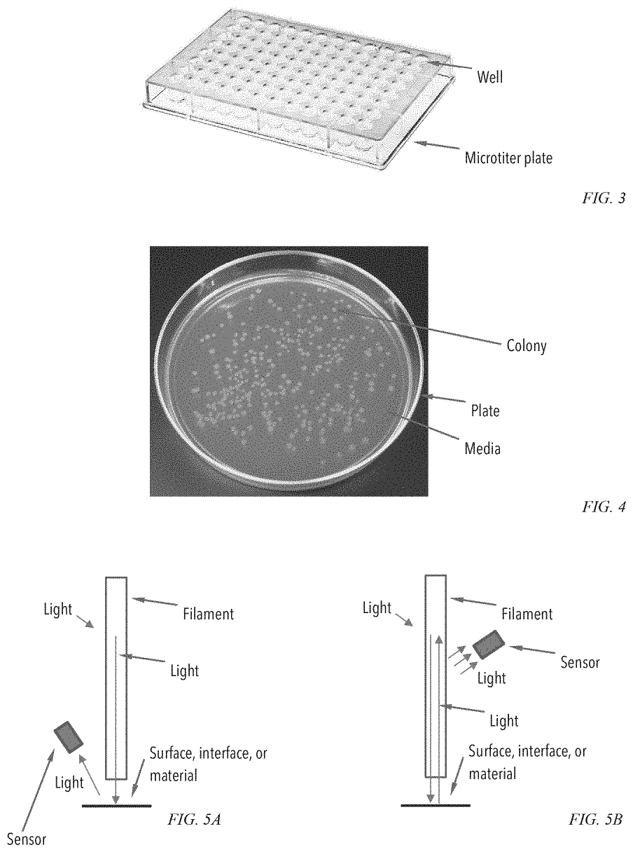

FIG. 3 illustrates a microtiter plate with multiple containers (“wells”) suitable for holding liquids, semisolids, semiliquids, colloids, emulsions, suspensions, slurries, or similar materials.

In at least one embodiment, an apparatus is used to collect material from a culture plate, a microtiter plate, a test tube or other liquid container, or from a container of some other kind. In at least one embodiment, an apparatus is used to collect material that is not in a container.

In at least one embodiment, an apparatus is used to collect material by bringing the material into contact with an end of a filament.

In at least one embodiment an apparatus is used to transfer material on an end of a filament. In at least one further embodiment, the aforementioned material on an end of a filament was collected on the end of the filament.

In at least one embodiment, material is collected onto an end of a filament by pressing a filament against or into or positioning a filament so as to contact a solid or semisolid or liquid material. In at least one embodiment, material is collected onto an end of a filament by submerging the end of the filament into a liquid or other material. In at least one embodiment, material is collected onto an end of a filament by submerging the end of the filament into a liquid or other material or contacting the surface of the liquid or other material, wherein a drop of liquid or other material adheres to the filament. In at least one embodiment, material is collected onto an end of a filament by submerging the end of the filament into a liquid, suspension, or emulsion or contacting the surface of a liquid, suspension, or emulsion, wherein particles suspended in the liquid, suspension, or emulsion adhere to the filament.

FIG. 3 illustrates a plate having containers formed into it, such containers sometimes referred to as wells by those skilled in the art. In at least one embodiment, a material in a well as in FIG. 3 or another suitable well or container is collected onto an end of a filament. FIG. 4 illustrates a culture plate containing culture media and microbial colonies. In at least one embodiment, one or more microbial colonies or portions of colonies are collected onto an end of a filament. In at least one further embodiment the aforementioned one or more microbial colonies or portions of colonies are configured in or on culture media in a culture plate, in a liquid, or otherwise.

Some parts of the description describe operations involving a single sample and/or a single well and/or a single culture plate. Some parts of the description describe operations involving multiple samples and/or multiple wells and/or multiple culture plates. One skilled in the art will appreciate that a microtiter plate or other device may contain more than one well such as without limiting the disclosure 96 wells or 384 wells. Thus, one skilled in the art will appreciate that, in at least one embodiment, multiple samples are applied to one or more wells in a microtiter plate or other container. Furthermore, in at least one embodiment, a single sample is applied to more than one well. Thus, in at least one embodiment, in at least one sequence of steps, at least one such step is applied once or multiple times to one or more samples and/or one or more wells, whereas at least one other such step is applied to a microtiter plate or other device as a whole or to a region of a plate as a whole, so said one other step is not specifically applied per sample or per well. One skilled in the art will appreciate that different embodiments of the present disclosure may be practiced using the same or different wells of one or more microtiter plates or other devices, while optionally the said one or more microtiter plates or other devices may have wells which are not used or which are used but do not constitute the practice of any embodiment herein. For example without limitation, a sample can be placed in two or more wells, and at least one well of said two or more wells is used according to a method comprising an embodiment of the present disclosure, while before, after, or in parallel, at least one other well of said two or more wells is used for some other purpose. Likewise, one embodiment can be practiced using at least one well on a microtiter plate or other device, and a different embodiment can practiced using at least one other well on the microtiter plate or other device.

In at least one embodiment, at least one location on a MALDI plate or other flat surface and at least one liquid containing location are used as part of one process to accomplish one or more than one analytical task.

A sample handling apparatus comprises in whole or part one or more elements as follows: an element, assembly, or apparatus described in US patent 5,063,791, titled “Sampling of Material” (William J. Martin), which is hereby incorporated herein by reference; an element, assembly, or apparatus described in patent applications GB878718232A and/or EP0307085A1, titled “Sampling of Material” (William J. Martin), which are hereby incorporated herein by reference; an element, assembly, or apparatus described in U.S. Pat. No. 4,613,573, titled “Automatic bacterial colony transfer apparatus” (Shibayama, et al.) which is hereby incorporated herein by reference; an element, assembly, or apparatus described in “Jubilee Demo: An Extensible Machine for Multi-Tool Fabrication” (Vasquez, Twigg-Smith, O'Leary, Peek. Jubilee Demo: An Extensible Machine for Multi-Tool Fabrication. CHI EA '20: Extended Abstracts of the 2020 CHI Conference on Human Factors in Computing Systems. April 2020 Pages 1-4 https://doi.org/10.1145/3334480.3383179), which is hereby incorporated herein by reference; an element or assembly of the Jubilee motion platform with Thingiverse ID 3843001 (https://www.thingiverse.com/thing:3843001) or github commit ID machineagency/jubilee/commit/76700425b644e68d5f71041afc3d75ebb93b6ec9 (https://github.com/machineagency/jubilee/commit/76700425b644e68d5f71041afc3d75ebb93b6ec9) and/or as described in the Hackaday article “Jubilee: A Toolchanging Homage To 3D Printer Hackers Everywhere” (Hackaday, Mike Szczys, Editor in Chief, https://hackaday.com/2019/11/14/jubilee-a-toolchanging-homage-to-3d-printer-hackers-everywhere/), which are hereby incorporated herein by reference, and/or any features of the associated Jubilee version 2.1.1 a motion platform and/or extruder; an element, assembly, or apparatus shown in one or more of FIGS. 2, 16, and/or 17; an element, assembly, or apparatus described in US patent application pub. no. 20190169560, titled “An Apparatus and a Method for Transferring Material” (Singer et al.), which is hereby incorporated herein by reference; an element, assembly, or apparatus comprising one or more of one, some, or all of the following elements: a filament positioning device movable in at least two dimensions, a bed optionally movable in at least one dimension, an optional storage area for filament in a fixed or movable position relative to the filament positioning device, an optional guide between a filament storage area and a filament positioning device, a filament cutting device which may be in a fixed or movable position relative to a filament positioning device, a camera, a filament cutter, a laser, a photodiode, a stage for a culture plate, a stage for a microtiter plate, a stage for a MALDI plate, a stager or holder for a test tube or for a liquid or other container of some other suitable type, an incubation chamber, a microcontroller or other suitable automated controller connected to and controlling appropriate elements of the apparatus, a strain or force sensor such as a piezoelectric or other sensor, a transducer for introducing vibrations such as a piezoelectric or other transducer, a positioning device for a culture plate, mictrotiter plate, and/or MALDI plate, a pipettor, and a matrix sprayer.

An apparatus as described in EMBODIMENT 101, any apparatus described herein, or some other appropriate apparatus is used to prepare a sample for MALDI analysis. A filament is used to apply at least one sample to at least one target position (or “spot”) of at least one MALDI plate. Optionally at least one time, a pipettor, matrix sprayer, filament, or other suitable method is used to apply a quantity of MALDI matrix and/or other material to the at least one target position, before or after the sample is applied to the target position. Optionally at least one time, a pipettor, MALDI sprayer, filament, or other suitable method is used to apply a quantity of at least one other reagent to the at least one sample on the at least one target position, before the sample is applied to the target position, after the sample is applied to the target position, or both before and after the sample is applied to the target position.

In at least one embodiment, a filament with a diameter of between about 0.2 mm and about 2.0 mm transfers material with a volume between approximately 0.2 μL and 5.0 μL to a target position of a MALDI plate. In at least one embodiment, a filament with a diameter of about 1.0 mm transfers material with a volume between approximately 0.5 μL and 1 μL to a target position of a MALDI plate.

An apparatus as described in EMBODIMENT 101, any apparatus described herein, or some other appropriate apparatus, is used to collect or transfer a quantity of material. In at least one embodiment, the quantity of material is a liquid. In at least one embodiment, the quantity of material is all or part of one or more microbial colony or predominantly composed of all or part of one or more microbial colony. In at least one embodiment, the quantity of material is neither a liquid nor a microbial colony, nor predominantly composed of a microbial colony.

In at least one embodiment, material is collected for the purpose of measuring a property of the material. In at least one embodiment, material is collected for the purpose of transporting the material. In at least one embodiment, material is not collected, and a filament is used to measure at least one property of a material without collecting a quantity of the material.

In at least one embodiment, material is collected from culture media and deposited on a surface or in a liquid. In at least one embodiment, material is collected from a liquid and deposited on a surface. In at least one embodiment, a quantity of material is collected from a material other than culture media or liquid and/or is deposited in or on a structure or substance other than a liquid or a surface.

An apparatus as described in EMBODIMENT 101, any apparatus described herein, or some other appropriate apparatus is used, in which light is transmitted through a filament, a mode of a filament, a mode of a filament and/or cladding, and/or a mode of a filament and/or cladding and surrounding atmosphere or other gas, liquid, fluid, or other substance.

In at least one embodiment, light is transmitted into and/or out of a filament such as for example without limitation an unclad PMMA fiber, by means of any combination of scattering, microscopic bending loss, macroscopic bending loss, or other optical property of the filament.

In at least one embodiment, light illuminates a side or end of a filament, is transmitted through the filament and emitted from an end or side of the filament, and subsequently illuminates an object, surface, interface, or substance on, near, or in a position capable of being illuminated from the filament.

In at least one embodiment, light illuminates an object, surface, interface, or substance on, near, or in a position capable of illuminating a filament and is reflected and/or transmitted into the filament, and subsequently is transmitted out of a side or end of the filament and illuminates a photosensor.

In at least one embodiment, light is transmitted into a side or end of a filament, subsequently illuminates an object, surface, interface, or substance, and light reflected or transmitted from the object, surface, interface, or substance subsequently illuminates a side or end of a filament, is transmitted out of a side or end of the filament, and illuminates a photosensor.

Those skilled in the art will appreciate that in at least one embodiment light is produced by at least one of a light emitting diode, a laser diode, another kind of laser, or another type of light source. Those skilled in the art will appreciate that in at least one embodiment, at least one optical device such as lenses, gratings, mirrors, filters, polarizers, apertures, and other types of optical components comprises a part of an apparatus, for the purpose of improving performance by gathering or focusing light, filtering out undesirable wavelengths or polarizations of light, or other optical functions familiar to those skilled in the art.

FIG. 5A illustrates a side view of a portion of an apparatus as described in EMBODIMENT 101, any apparatus described herein, or some other appropriate apparatus, wherein a filament is positioned in proximity to a surface, interface, or other material. Light illuminates the side of the filament, is transmitted through the filament and emitted from an end of the filament, and subsequently illuminates a surface, interface, or other material. Light is reflected and/or otherwise re-emitted from the surface, interface, or other material and detected by the sensor. The intensity, polarization, and/or frequency of the re-emitted light is detected by a photodetector or similar sensor, and depends on the distance between the end of the filament and the surface, interface, or other material and the physical properties of the surface, interface, or other material. Thus, the intensity, polarization, and/or frequency of the re-emitted light can be used to determine the distance between the end of the surface, interface, or other material and/or the physical properties of the surface, interface, or other material.

FIG. 5B illustrates a portion of an apparatus as described in EMBODIMENT 101, any apparatus described herein, or some other appropriate apparatus, wherein a filament is positioned in proximity to a surface, interface, or other material. Light illuminates the side of the filament, is transmitted through the filament and emitted from an end of the filament, and subsequently illuminates a surface, interface, or other material. Light is reflected and/or otherwise re-emitted from the surface, interface, or other material and illuminates the end of the filament and is transmitted through the filament. Light is emitted from the side of the filament and detected by the sensor. The intensity, polarization, and/or frequency of the emitted light is detected by a photodetector or similar sensor, and depends on the distance between the end of the filament and the surface, interface, or other material and the physical properties of the surface, interface, or other material. Thus, the intensity, polarization, and/or frequency of the light emitted from the side of the filament can be used to determine the distance between the end of the surface, interface, or other material and/or the physical properties of the surface, interface, or other material.

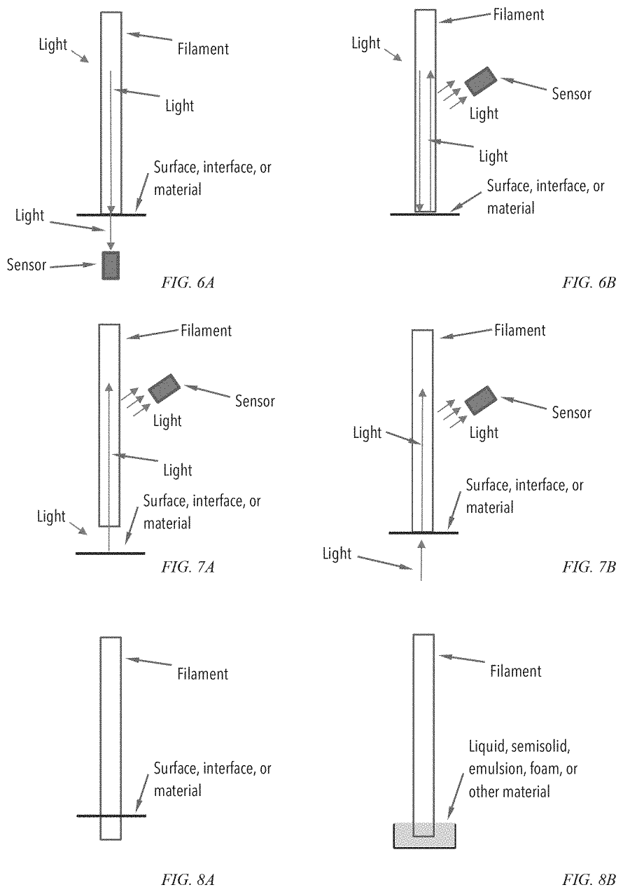

FIG. 6A illustrates a portion of an apparatus as in FIG. 5A, except that light is transmitted through a surface, interface, or other material rather than being reflected.

FIG. 6B illustrates a portion of an apparatus as in FIG. 5B, except that an end of a filament is contacting or nearly contacting a surface, interface, or other material rather than being in proximity.

FIG. 7A illustrates a portion of an apparatus as in FIG. 5A, except that the light path is reversed. Light illuminates a surface, interface, or other material. Light is reflected and/or otherwise re-emitted from the surface, interface, or other material and illuminates the end of the filament and is transmitted through the filament. Light is emitted from the side of the filament and detected by the sensor. The intensity, polarization, and/or frequency of the emitted light is detected by a photodetector or similar sensor, and depends on the distance between the end of the filament and the surface, interface, or other material and the physical properties of the surface, interface, or other material. Therefore, the intensity, polarization, and/or frequency of the light emitted from the side of the filament can be used to determine the distance between the end of the surface, interface, or other material and/or the physical properties of the surface, interface, or other material.

FIG. 7B illustrates a portion of an apparatus as in FIG. 7A, except that light is transmitted through a surface, interface, or other material rather than being reflected.

FIG. 8A illustrates a portion of an apparatus as in any of FIGS. 5A-7B, wherein a filament has penetrated a surface, interface, or other material.

FIG. 8B illustrates a portion of an apparatus as in any of FIGS. 5A-7B, wherein a filament end is submerged in a liquid, semisolid, emulsion, foam, or other material.

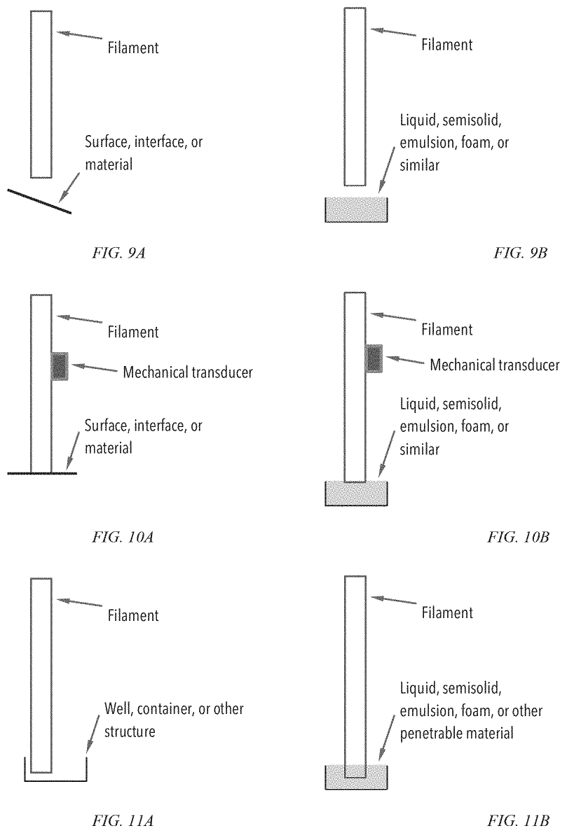

FIG. 9A illustrates a portion of an apparatus as in any of FIGS. 5A-8B or 9B or any other appropriate apparatus described herein, wherein a surface, interface, or other material is not orthogonal to a filament.

FIG. 9B illustrates a portion of an apparatus as in any of FIGS. 5A-7B or any other appropriate apparatus described herein, except that a filament in in proximity to but not immersed in a liquid semisolid, emulsion, foam, or other material.

An apparatus as described in EMBODIMENT 101, any apparatus described herein, or some other appropriate apparatus is used, in which contact with a surface, interface, or material is detected or measured by means applying a mechanical force to a filament and observing the mechanical response of the filament by means of a force transducer, strain gauge, an optical apparatus as described elsewhere herein, a measurement of the motor current required to move the filament, a measurement of the deflection of the filament, or any other appropriate means. In at least one embodiment, force is applied to a filament by moving the filament in any or all of the X, Y, and Z directions with a filament positioning device. In at least one embodiment, force is applied to a filament by applying force with a mechanical transducer such as without limitation a piezoelectric transducer.

In at least one embodiment, a filament is induced to resonate as in a cantilever beam, the resonant frequency of the filament measured by the same or a different mechanical transducer or by another means described herein, and contact with a surface, interface, or material is detected or measured as a change in resonant frequency and/or amplitude as the free end of the filament makes contact with the surface, interface, or material.

FIG. 10A illustrates a portion of an apparatus as described in EMBODIMENT 101, any apparatus described herein, or some other appropriate apparatus, wherein a filament is positioned in proximity to a surface, interface, or other material. Motion is induced in the filament as described in this embodiment or in another embodiment herein, and the filament is brought into contact with the surface, interface, or other material by any means described herein. As the filament contacts the surface, interface, or other material, a change in force, resistance, resonant frequency, position, or other mechanical property is detected as described in this embodiment or in another embodiment herein.

FIG. 10B illustrates a portion of an apparatus as in FIG. 10A, wherein a filament is positioned in proximity to a liquid, semisolid, emulsion, foam, or similar material.

FIG. 11A illustrates a portion of an apparatus as in FIG. 10A, wherein a filament is positioned in proximity to a well, container, or other structure with a wall, shoulder, lip, or edge, and using any means described herein the filament is brought into contact with the well, container, or other structure.

FIG. 11B illustrates a portion of an apparatus as in FIG. 10B, wherein a filament end is submerged in a liquid, semisolid, emulsion, foam, or similar material. The change in force, resistance, or resonant frequency or amplitude of a filament depends on the depth of submersion and on the viscosity, density, thixotropy, other rheology, and other properties of the liquid, semisolid, emulsion, foam, or similar material. Further, if the end of the filament is withdrawn above the original surface height of the liquid, semisolid, emulsion, foam, or similar material, the change in force, resistance, or resonant frequency or amplitude of a filament depends on the height above the original surface and on the viscosity, density, thixotropy, other rheology, and other properties of the liquid, semisolid, emulsion, foam, or similar material. Consequently, if some of the aforementioned properties are known, then one or more of the aforementioned measurements can determine one or more of the aforementioned properties that are unknown. For example without limitation, in at least one embodiment, a filament is inserted into and withdrawn from a liquid while being induced to vibrate as a cantilever beam, the change in resonant frequency with relative height of the filament end indicating viscosity of the liquid. By way of another example, in at least one embodiment, an apparatus as described herein is used as an acoustic rheometer.

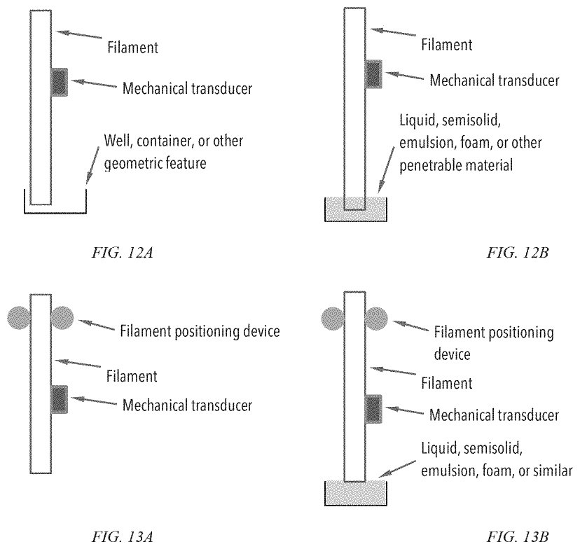

FIG. 12A illustrates a portion of an apparatus as in FIG. 11A, wherein at least one transducer is part of the apparatus and is used for at least one of applying force, inducing motion, or sensing force, strain, or motion.

FIG. 12B illustrates a portion of an apparatus as in FIG. 11B, wherein at least one transducer is part of the apparatus and is used for at least one of applying force, inducing motion, or sensing force, strain, or motion.

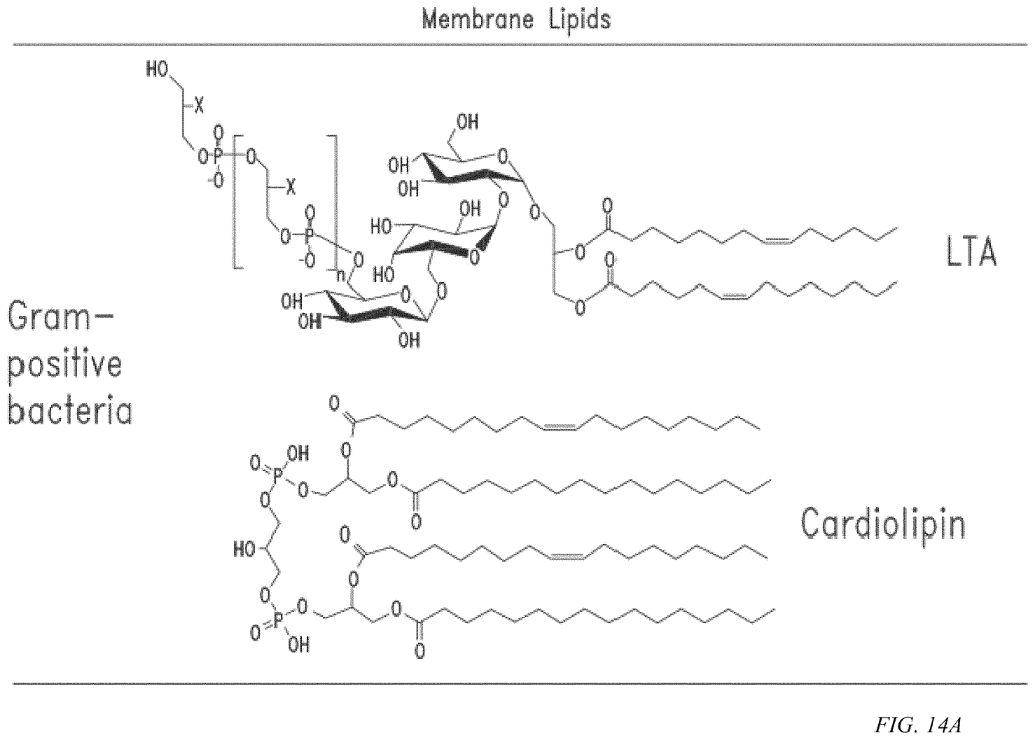

FIG. 13A illustrates a portion of an apparatus as in FIGS. 8A-12B, wherein a filament positioning device restrains the motion of a filament in the manner of a cantilever.

FIG. 13B illustrates a portion of an apparatus as in FIGS. 10B, 11B, 12B, and 13A, wherein a filament positioning device restrains the motion of a filament in the manner of a cantilever.

An apparatus as described in EMBODIMENT 101, any apparatus described herein, or some other appropriate apparatus, is used to position a filament and/or an end of a filament. In at least one embodiment, a filament and/or an end of a filament is positioned to collect a material. In at least one embodiment, a filament and/or and end of a filament is positioned to measure a property of a material. In at least one embodiment, a filament and/or and end of a filament is positioned to collect a material and measure a property of a material.

In at least one embodiment, a camera is used to measure or estimate the height of a surface of an object and/or a location of a feature or target on an object, and a filament is then positioned by means of a filament positioning device and/or one or more axes of motion moving for example the filament positioning device and/or a bed to position an end of a filament relative to the location of the feature or target on the object. In at least one embodiment, multiple camera images are used to estimate the height of a surface of an object and/or a location of a feature or target on an object for example by means of focus stacking.

In at least one embodiment, a method as described herein is used to measure or estimate a property of a sample on, in proximity to, in view of, or capable of being accessed by a filament end, to determine a property of the sample or material. For example without limitation in at least one embodiment at least one of the property of mass, presence, viscosity, fluorescence, viability, growth, volume, species, hardness, concentration, turbidity, or birefringence is measured or estimated.

FIG. 16 illustrates a filament cartridge. The filament cartridge comprises a hollow box, in which a filament is situated as a coiled loop. The filament passes through a nipple into a port at one side of the filament cartridge, then exits the filament cartridge. A removable cap covers the port. The nipple is designed to prevent or impede substances outside the filament cartridge from infiltrating to the inside of the filament cartridge. The filament cartridge is made in an upper and a lower piece, joined by a seam. The filament is placed inside the lower piece then threaded through the nipple in the upper piece, then the seam is welded or otherwise sealed. Those skilled in the art will appreciate that a filament cartridge having a different process of assembly and/or different components can achieve the equivalent result of the cartridge shown in FIG. 16, and in at least one embodiment, an apparatus is used that contains a filament cartridge is used to store and dispense filament, said cartridge having a different process of assembly and/or different components than the filament cartridge shown in FIG. 16.

Filament cartridges such as shown in FIG. 16 are commonly used to store and dispense surgical sutures, particularly for veterinary use. The filament and the inside of the filament cartridge are sterilized. The nipple prevents or impedes non-sterile material from entering the filament cartridge. The cap covers the port, preventing or reducing contamination of the filament in the port, the inside of the port, and the nipple. In at least one embodiment, an apparatus is used that contains a filament cartridge containing a surgical suture. In at least one other embodiment, an apparatus is used that contains a filament cartridge containing a filament that is not a surgical suture.

In at least one embodiment, a filament cartridge is a filament storage area. In at least one embodiment, an apparatus is used that contains a filament cartridge that is not mounted on a filament positioning device but instead is mounted in some other position, for example without limitation a filament cartridge is mounted in a stationary position in the apparatus.

FIG. 16 illustrates a filament cartridge mounted on a filament positioning device. The filament cartridge is mounted with the port of the filament cartridge facing towards the filament positioning device. A filament in the filament cartridge passes out of the port into a filament drive, then optionally passes through or along a filament stiffening guide. The filament drive is driven by a filament drive motor. In at least one embodiment, an apparatus as described herein includes a filament positioning device, and furthermore a filament cartridge is mounted on the filament positioning device or other element of the apparatus, wherein a filament in the filament cartridge passes out of the cartridge and into a filament drive, then optionally through or along a filament stiffening guide.

An apparatus as shown in FIG. 16 allows any of a variety of sterile filaments contained in cartridges currently available from multiple vendors to be used with a method described herein. An apparatus as shown in FIG. 16 has the desirable properties of allowing filament to be easily mounted on a filament positioning device, of facilitating maintaining the sterility of a filament dispensed from a cartridge, and of allowing filaments of different diameters or other properties to be swapped on a filament positioning device while maintaining sterility and furthermore allowing said filaments of different diameters or other properties to be stored after partial use and removal from the aforementioned apparatus, then at a later time re-mounted and further used.

FIG. 17 illustrates a filament positioning device. A filament passes into or otherwise comes in contact with a filament drive, wherein the filament contacts at least one wheel that is part of the filament drive. At least one wheel of the filament drive is a driven wheel and is rotated by a filament drive motor, which causes the least one driven wheel to move the filament forwards and/or backwards through the filament drive. By the static resistance of the filament drive motor and/or by applying power to the filament drive motor in such a way as to resist rotation, the filament drive motor causes the at least one driven wheel to prevent the filament from moving forwards or backwards relative to the filament drive except when the filament drive motor rotates the at least one driven wheel under power. As a consequence of the actions of the filament drive motor and the driven wheel, the filament drive positions a filament along a vertical or non-vertical axis, holds the position of the filament along a vertical or non-vertical axis, and draws filament for example from a filament storage location optionally through a guide to the filament positioning device.

The filament passes from the filament drive into an optional filament stiffening guide, which allows the filament to be positioned with greater accuracy and/or allows the filament to press or penetrate objects with greater force than an equivalent apparatus without a filament stiffening guide. The distal end of the filament can extend past the distal end of the filament stiffening guide for contacting substances and/or to perform collection and/or transport of substances and/or to perform any appropriate method described herein. In at least one operation of at least one embodiment, an end of the filament regarded with reference to FIG. 17 as the distal end of the filament is positioned at the location of or approximately at the location of the distal end of the filament stiffening guide. In at least one operation of at least one embodiment, an end of the filament is positioned at a location within the filament positioning guide, so that the end of the filament does not extend past the distal end of the filament positioning guide, for the purpose of shielding the end of the filament and/or a length of the filament from stray light, preventing and/or reducing light from being transmitted out of the filament, focusing or reflecting light into and/or out of the filament, another optical effect other than shielding, preventing transmission, focusing, or reflecting, or for effecting and/or improving the operation of any method described herein. In one embodiment, an apparatus is used that contains a filament stiffening guide that is a hollow round tube composed of steel, a metal other than steel, and/or a material other than metal. In at least one embodiment, an apparatus is used that contains a filament stiffening guide that is not a hollow tube and/or has a bore cross-section that is not circular. In one embodiment, the proximal end of the filament stiffening guide is located close to a filament drive, such that the length of a filament passing from the filament drive to the filament stiffening guide is relatively short, thereby preventing or reducing flexing of the filament between the filament drive and the filament stiffening guide, such flexing being undesirable in at least one embodiment by reducing the force with which a filament can contact and/or penetrate a surface. In at least one embodiment, locating the proximal end of a filament stiffening guide close to a filament drive allows a filament fed from the filament drive to pass into the filament stiffening guide without additional guiding or other operation to facilitate passage of the filament into the filament stiffening guide. In at least one embodiment, a structure is placed between a filament drive and the proximal end of a filament stiffening guide, said structure having the effect of guiding an end of a filament into the filament stiffening guide as the filament is fed out of the filament drive. In at least one embodiment, the aperture of the proximal end of a filament stiffening guide is flared or otherwise larger than the bore of the filament stiffening guide and/or is fitted with at least one funnel or other guiding structure, with the effect that an end of a filament is guided into the filament stiffening guide as the filament is fed out of the filament drive.

In at least one embodiment, a filament positioning device such as without limitation as shown in FIG. 17 is moved in the X, XY, or XYZ dimensions by for example being mounted on a tool carrier attached to a linear movement.

EMBODIMENT 101: Extraction of Microbial Lipids

1. Using a method described herein, place a sample on a spot of a MALDI plate or similar device. The sample may be a microbial colony, a liquid sample, or some other sample. The sample may have a volume of less than about 0.5 μL, about 0.5 μL, about 1.0 μL, about 1.5 μL, about 2 μL, about 3 μL, about 4 μL, about 6 μL, about 8 μL, about 10 μL, about 20 μL, or greater than 20 μL. Optionally allow the sample to dry or partially dry. In at least one embodiment, at least one reagent or other material is pre-applied to at least one spot of the plate before a sample is placed on said at least one spot.

2. Optionally, using a method described herein or another suitable method known to those skilled in the art, place a reagent or other material on the aforementioned spot. For example without limitation, place 1 μL of a solution of citric acid and sodium citrate on a spot. Optionally allow the sample to dry or partially dry.

3. Optionally heat the plate. In at least one embodiment, the plate is heated to less than 95° C., about 95° C., about 100° C., about 110° C., about 121° C., about 125° C., about 130° C., about 135° C., or more than 135° C. In at least one embodiment the plate is heated to above 121° C. In at least one embodiment, the plate is heated for less than 15 min., about 15 min., about 20 min., about 25 min., about 30 min., or longer than 30 min.

In at least one embodiment, a plate is prevented from completely drying while it is heated by heating the plate in a humid atmosphere. In at least one embodiment, at least one spot of a plate is prevented from partly or completely drying while it is heated by supplying moisture periodically or continuously to the at least one spot on the plate by a method described herein or another suitable method known to those skilled in the art. In some embodiments, the plate is heated in an enclosed space to prevent or minimize evaporation from the surface of the plate.

4. Optionally wash the plate. In at least one embodiment, a plate is washed by at least one application of at least one liquid, using a method described herein or another suitable method known to those skilled in the art. In at least one embodiment, at least one liquid is water. In at least one embodiment, at least one liquid is a composition containing at least one of: water, a detergent, an alcohol, an emulsifier, or an organic solvent, including but not limited to: phenol, chloroform, methanol, ethanol, etc. After at least one application of at least one liquid, optionally allow the plate to dry or partially dry.

One skilled in the art will appreciate that, in at least one embodiment the method of EMBODIMENT 101 extracts molecules that are not lipids in addition to or instead of extracting lipids. EMBODIMENT 101 is also referred to as “a method for extracting lipids.” As used herein “extraction” and “isolation” are used interchangeably to mean the removal of molecules from a sample.

It will be understood in the art that the methods and parameters for extracting lipids may differ for different microbial organisms, some may require additional growth time, and different membrane characteristics will affect extraction. Based on this disclosure, it is within the ordinary level of skill in the art to determine appropriate uses and quantities of solvents, detergents, buffers, heating setting, etc. to carry out the methods of the present disclosure.

EMBODIMENT 102: Mass Spectrometric Analysis

1. Extract lipids according to EMBODIMENT 101.

2. Optionally apply a MALDI matrix to some or all of the spots on which samples have been placed. Allow the plate to dry as necessary.

3. Place the plate in a mass spectrometer or otherwise present some or all of the contents of one or more spots to a mass spectrometer and collect mass spectra from the one or more spots. In at least one embodiment, a single spectrum is collected from one or more spots. In at least one embodiment, multiple spectra are collected from one or more spots. In at least one embodiment, multiple spectra with different m/z (mass to charge ratio) ranges are collected from at least one spot. In at least one embodiment at least one spectrum is collected with a lowest m/z of about less than 100, 100, 300, 600, 700, 800, or 1000 m/z. In at least one embodiment a spectrum is collected with a lowest m/z of greater than 1000 m/z. In at least one embodiment a spectrum is collected with a highest m/z of about 800, 1000, 1200, 1500, 1800, 2000, 2200, 2400, or 2500 m/z. In at least one embodiment a spectrum is collected with a highest m/z of higher than 2500 m/z. In at least one embodiment, two or more spectra are collected that differ in mass spectrometer parameters other than mass range, such as without limiting the disclosure, detector gain.

In at least one embodiment, a mass spectrometer is used that is an electrospray mass spectrometer, a desorption electrospray ionization (DESI) mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (MALDI) mass spectrometer, a MALDI imaging mass spectrometer, a Time-of-Flight Secondary Ion Mass Spectrometry (TOF-SIMS) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using a nebulization-ionization method such as surface acoustic wave nebulization (SAWN), a mass spectrometer using Rapid Evaporative Ionization Mass Spectrometry, a surface acoustic wave (SAWN) mass spectrometer, or another type of mass spectrometer.

In at least one embodiment, a mass spectrometer or an inlet or inlet capillary or other sub-assembly of a mass spectrometer is positioned relative to a sample by a motion platform.

EMBODIMENT 103: Spectroscopic Analysis

1. Extract lipids according to EMBODIMENT 101.

2. Analyze the plate with a spectroscopic instrument, where said instrument operates by laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, flow cytometry, or some other type of spectroscopy; or by means of a scintillation detector, scintillation counter, Geiger counter, ionization chamber, gaseous ionization detector, or other radiation detector;

In at least one embodiment, a spectroscope, an inlet or inlet capillary or other sub-assembly of a mass spectrometer, an illuminating element of a spectroscope, a light detecting element of a spectroscope, or a fiber and/or other optics for directing light to and/or from a spectroscope is positioned relative to a sample by a motion platform.

EMBODIMENT 104: Combined Analysis

Collect both mass spectrometric and spectroscopic information on a sample, by performing at least one of the following steps.

1. Perform the steps of both EMBODIMENT 102 and EMBODIMENT 103. In at least one embodiment, EMBODIMENT 102 is performed before EMBODIMENT 103, EMBODIMENT 103 is performed before EMBODIMENT 102, or EMBODIMENTS 102 and 103 are performed in parallel. In at least one embodiment, EMBODIMENT 102 and EMBODIMENT 103 are performed on one or more spots on the same plate. In at least one embodiment, at least two spots on the same or on different plates are used for performing at least one of EMBODIMENT 102 or EMBODIMENT 103.

2. Perform the steps of EMBODIMENT 102, then using the same plate perform the steps of EMBODIMENT 103 beginning with step 2 of EMBODIMENT 103.

3. Perform the steps of EMBODIMENT 103, then using the same plate perform the steps of EMBODIMENT 102, beginning with step 2 of EMBODIMENT 102.

EMBODIMENT 105: Classification of Spectra

1. Optionally preprocess at least one spectrum. For example without limiting the disclosure, in at least one embodiment, at least one spectrum is preprocessed by at least one of baseline correction, alignment, charge state deconvolution, isotopic deconvolution, Fourier transform, a type of integral transform other than Fourier transform, peak extraction, or some other kind of feature extraction. In at least one embodiment, a sequence of preprocessing operations is performed on one or more spectra and at least one additional sequence of preprocessing operations is performed on the one or more spectra and/or on one or more additional spectra, where each the aforementioned spectra is obtained from a distinct sample or alternatively at least two of the aforementioned spectra are obtained from the same sample.

2. Classify at least one spectrum using a classification method.

For example without limiting the disclosure, in at least one embodiment at least one spectrum is classified by comparing the spectrum to an exemplar, average, consensus, or synthetically generated spectrum from a library. As a further example, in at least one embodiment, at least one spectrum is classified partly or entirely without comparison to another spectrum. For example without limiting the disclosure, in at least one embodiment at least one spectrum is classified using a machine learning method such as support vector machines. One skilled in the art will appreciate that certain machine learning methods are typically performed using machine learning models that have previously been trained on training data with properties related to the data to be classified.

In at least one embodiment, classification is performed on a single spectrum or on features extracted from a single spectrum from at least one sample. In at least one embodiment, classification is performed on at least two spectra or on features extracted from at least two spectra. In at least one embodiment, the aforementioned at least two spectra are from the same spot on the same plate. In at least one embodiment, a first spectrum of at least two spectra is from a first spot on a first plate whereas a second spectrum of the at least two spectra is from a second spot on the first plate or on a second plate. In at least one embodiment, three or more spectra from one or more spots are used. In at least one embodiment, at least two spots are processed under essentially the same conditions. In at least one embodiment at least two spots are processed differently, using any or all of at least the various methods discussed in the following embodiments, including without limitation use of buffers with different compositions and/or in different amounts, or use of lysozyme in different amounts.

In at least one embodiment, at least one spectrum used for training data and/or as an exemplar, average, or consensus spectrum is produced by a method described in one of the embodiments or elsewhere herein. In at least one embodiment, at least one spectrum used for training data and/or exemplar, average, or consensus spectra is produced by some other method besides a method described in one of the embodiments or elsewhere herein.

EMBODIMENT 106: Classification of Samples

1. Obtain at least one spectrum from at least one sample by performing the steps of EMBODIMENTS 102, 103, or 104.

2. Classify samples by performing the steps of EMBODIMENT 105 one or more times on the at least one spectrum obtained in step 1.

In at least one embodiment, at least one sample is classified hierarchically. The at least one sample is first classified at a more general level by performing the steps of EMBODIMENT 105 for a first classification, then the at least one sample is classified to a more specific level by performing the steps of EMBODIMENT 105 again for a second classification; wherein either the same spectrum is used for the aforementioned first classification and second classification or else at least one spectrum is used for the first classification and not the second classification, and/or at least one spectrum is used for the second classification and not the first classification. Furthermore, in at least one embodiment, the aforementioned hierarchical classification is extended in like manner to a third classification step or to any number of classification steps.

EMBODIMENT 107: Screen Samples

1. According to the steps of EMBODIMENT 106, using at least one spectrum, classify at least one sample for the presence of microbes or for the presence of microbes above a threshold amount, or conversely classify at least one sample for the absence of microbes or for the absence of microbes below a threshold amount, or classify at least one sample for the presence or absence or for the presence or absence above or below a threshold amount of a taxon of microbe such as without limitation bacteria, or for the presence or absence or for the presence or absence above or below a threshold amount of one or more categories not corresponding to taxon, such as without limitation Gram stain. In an embodiment, at least one spectrum is a Raman spectrum.

2. If in step 1 the aforementioned sample is determined to have microbes of interest present or present above a threshold value, then according to the steps of EMBODIMENT 106, optionally using different spectra from step 1, classify the sample according to at least one of microbial species, strain, taxon above the level of species, antimicrobial susceptibility or resistance, virulence, and/or one or more other categories. In an embodiment, step 2 is performed using at least one additional spectrum, and the at least one additional spectrum is a MALDI spectrum.

For example without limitation, in an alternative embodiment, a Raman spectrum of a sample determines the presence or absence of microbes belonging to one or more classes in the sample, said one or more classes either corresponding to taxa or not corresponding to taxa, then a MALDI mass spectrum determines the identity of microbes present at a greater refinement than was determined with the Raman spectrum, such as without limitation microbial species, strain, taxon above the level of species, antimicrobial susceptibility or resistance, or virulence.

EMBODIMENT 108: Classify Samples Via Mass Spectrometry

According to the steps of EMBODIMENT 106, using at least one mass spectrum, classify a sample according to at least one of microbial species, strain, taxon above the level of species, strain, antimicrobial susceptibility or resistance, virulence, and/or one or more other categories.

EMBODIMENT 109: Classify Samples Via Mass Spectrometry

According to the steps of EMBODIMENT 106, using at least one MALDI mass spectrum or a mass spectrum that is not a MALDI mass spectrum, classify a sample according to at least one of microbial species, strain, taxon above the level of species, strain, antimicrobial susceptibility or resistance, virulence, and/or one or more other categories.

EMBODIMENT 110: Classify Samples Via Spectroscopy

According to the steps of EMBODIMENT 106, using at least one spectrographic spectrum, classify a sample according to at least one of microbial species, strain, taxon above the level of species, strain, antimicrobial susceptibility or resistance, virulence, and/or one or more other categories.

EMBODIMENT 111: Classify Samples Via Raman Spectroscopy According to the steps of EMBODIMENT 106, using at least one Raman spectrum, classify a sample according to at least one of microbial species, strain, taxon above the level of species, strain, antimicrobial susceptibility or resistance, virulence, and/or one or more other categories.

EMBODIMENT 112: Clinical Sample

1. Any of the steps of EMBODIMENTS 101-111 are performed, using at least one sample that is a biological sample and/or a clinical sample.

In at least one embodiment, the biological sample is derived from a clinical specimen or sample. In at least one embodiment, at least one sample is obtained from a urine specimen, a blood sample, a sample incubated in a blood bottle, sputum, a sample obtained from sputum, feces, wound effluent, mucus, buccal swab, nasal swab, vaginal swab, nipple aspirate, sweat, saliva, semen or ejaculate, synovial fluid, bronchoalveolar lavage, tears, a urinary catheter sample, a culture plate, or another clinical or medical sample.

EMBODIMENT 113: Non-Clinical Sample

1. Any of the steps of EMBODIMENTS 101-111 are performed, using at least one sample that is a not a clinical sample.

In at least one embodiment, the non-clinical sample is an industrial sample, an environmental sample, an agricultural sample, a veterinary sample, a food sample, a forensic sample, a manufacturing process sample, a fermentation sample, a sterility sample, or any other sample potentially containing a microbial organism.

EMBODIMENT 114: Culture Smear with Acid

Perform the steps of EMBODIMENT 101 as follows:

In step 601, obtain a flat plate such as a MALDI plate, on which an acidic material such as dry citric acid has been deposited on one or more spots. Alternatively, obtain a plate and deposit an acidic material on one or more spots of the plate. In at least one embodiment, a liquid solution is or has been deposited and allowed to partly or entirely dry, leaving behind an acidic material. Place the plate on a bed of an apparatus.

In step 602, using a method described herein, apply a sample comprising at least a portion of one microbial colony from at least one culture plate onto at least one spot of the plate. For example without limitation, use a camera locate the position and height of at least one microbial colony in at least one petri dish or other culture plate on a bed, then locate a filament positioning device holding a filament to collect and transfer the microbial colony from the petri dish or other culture plate to the flat plate.

In step 603, heat the flat plate. In at least one embodiment the flat plate is heated by heating the bed. In at least one embodiment, a cover is placed over the flat plate and/or the bed to retain moisture during heating, and/or moisture is added to the environment of the plate prior to and/or during heating. In at least one embodiment, water or other liquid is transferred to locations on the flat plate during heating using a filament or by another method described herein.

In an embodiment, the flat plate is heated to 110° C. for 30 minutes or 121° C. for 30 minutes.

In step 604, remove the cover, if any. Optionally allow the plate to cool. Optionally wash the plate with a liquid. In an embodiment, the plate is washed with water transferred by a filament or by any method described herein.

In step 605, optionally apply a MALDI matrix in a solution or suspension to at least one spot on the plate, using a filament, a pipette, a matrix sprayer, or any method described herein. Allow to dry. In an embodiment, the matrix is norharmane. In an embodiment, the matrix is dissolved in a mixture containing chloroform and methanol.

EMBODIMENT 115: Culture Smear with Buffer

Perform the steps of EMBODIMENT 114, except in step 601 a buffer solution is used in place of acid. In at least one embodiment, the steps of EMBODIMENT 114 are performed, except that in step 601 in place of acid apply about 1 μL of a solution containing citric acid at about 0.3M concentration and sodium citrate at about 0.3M concentration.

EMBODIMENT 116: Culture Smear with no Acid or Buffer

Perform the steps of EMBODIMENT 114, except in step 601 no acid or buffer has been or is deposited.

EMBODIMENT 117: Escherichia coli and Maldi

Perform the steps of EMBODIMENT 114.

Place the flat plate in a MALDI mass spectrometer. Collect a spectrum from 1000 m/z to 2400 m/z in negative ion mode.

EMBODIMENT 118: Membrane Lipids

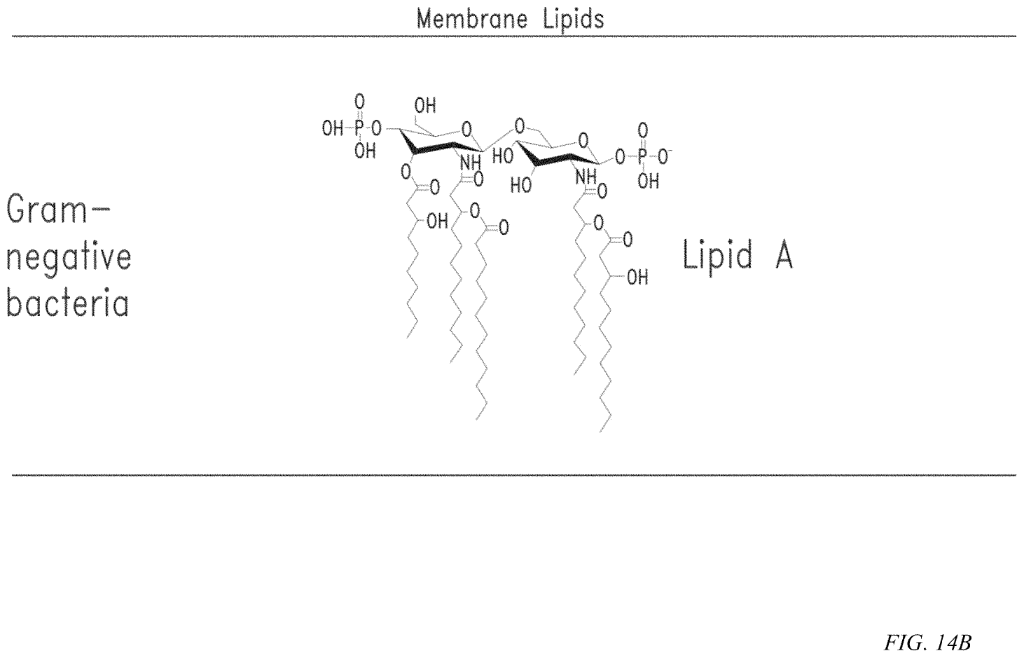

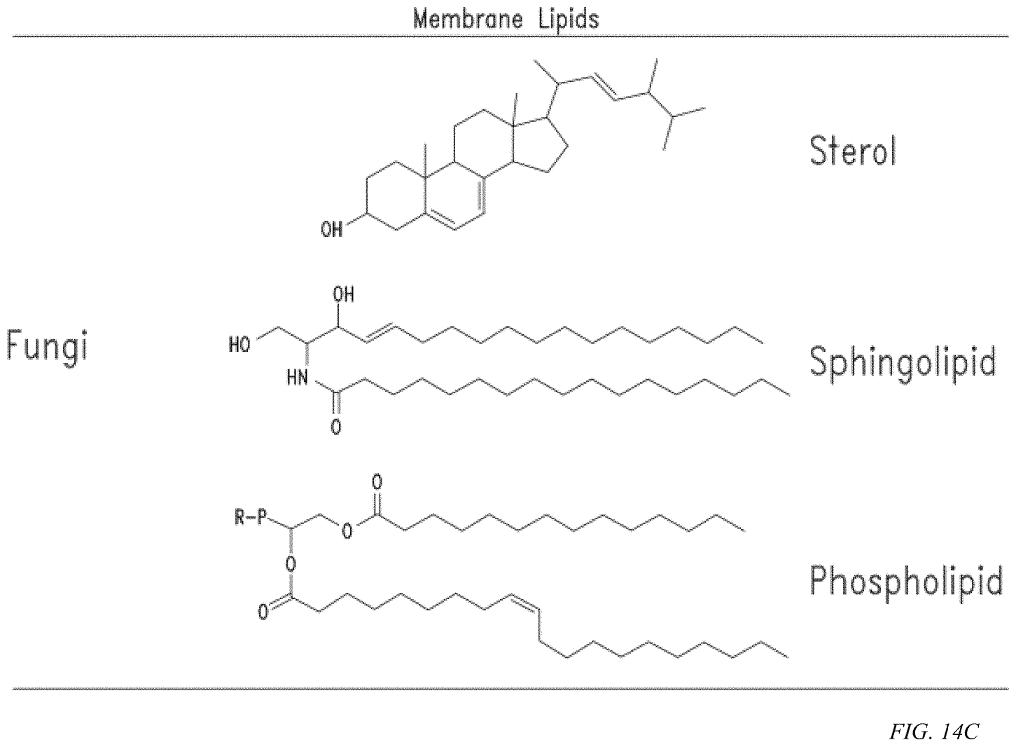

FIGS. 14A through 14C depict example lipid types for three classes of microbes: Gram-positive bacteria, Gram-negative bacteria, and fungi. In at least one embodiment, for said classes of microbes, for at least one of said class of microbe, at least one type of said example lipid types is extracted. In at least one embodiment, for said classes of microbes, for at least one of said class of microbe, at least one lipid is extracted that is not shown in FIGS. 14A through 14C as an example lipid type for said class of microbe.

EMBODIMENT 119: Lipid A

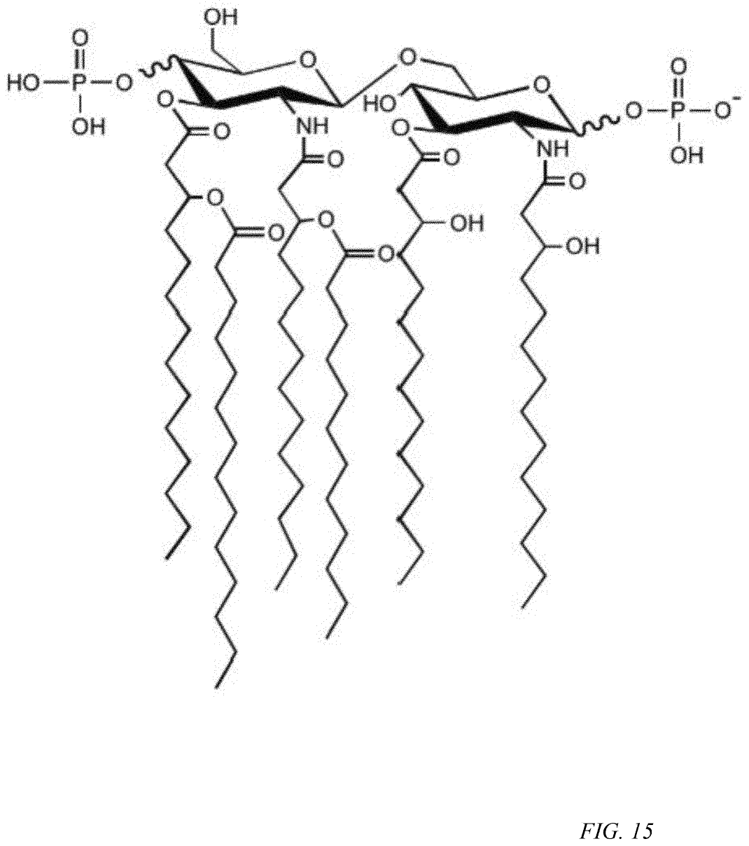

FIG. 15 depicts an example structure of lipid A. This structure is associated in the literature with lipid A from E. coli, observed at a nominal mass of about 1798 Da.

EMBODIMENT 120: Kits

In at least one embodiment, at least one kit is distributed, said kit consisting in part or whole of one or more instances of at least one of the following: a plate, a plate with material pre-dispensed on it using any method described herein or by any other method, a gasket as described herein or of another type, a buffer solution, an acidic solution, a solid to which a liquid can be added to form a buffer or acidic solution, a matrix solution, a solid to which a liquid can be added to form a matrix solution, instructions, and a software program and/or license key and/or other rights or credentials to a software program or software service that with or without additional data classifies samples and/or spectra according to any of the embodiments and/or any of the methods described herein.

In at least one embodiment, a kit contains at least one object not mentioned in the preceding description of EMBODIMENT 120.

EMBODIMENT 121: Antimicrobial Susceptibility Test

In at least one embodiment, at least one microbial colony or other sample is tested for antimicrobial susceptibility and/or antimicrobial resistance by means of (a) incubating or culturing the at least one sample in at least one volume of culture media containing a concentration of an antimicrobial agent, producing at least one inoculum; (b) preparing the at least one inoculum for analysis, including without limitation using any method described herein.

In at least one embodiment, at least one part of a process of a test for antimicrobial susceptibility and/or antimicrobial resistance is performed using an apparatus as described herein and/or using a method described herein.

EMBODIMENT 122: Spatial Information

Perform at least one of the following steps:

1. Perform the method of one or more of EMBODIMENTS 102, 103, 104, 105, or 106. However, in addition to spectral information, capture information about the physical location on the plate from which one or more spectra were obtained.

2. Perform the method of one or more of EMBODIMENTS 102, 103, 104, 105, or 106. Obtain spectra from multiple locations on a spot, and segregate spectra by location on the spot, by one or more of at least the methods of combing only spectra that were collected at the same or substantially the same location, clustering spectra by location, and clustering spectra by relative similarity.

3. Perform the method of one or more of EMBODIMENTS 101-106 or 108-111. In at least one embodiment, at least two affinity reagents are placed in two or more respective spots, or else two or more of the at least two affinity reagents are placed in a single spot and any remaining affinity reagents are placed in the same or different spots.

In at least one embodiment, for at least one lipid or other analyte of interest, a pattern of distribution is detected. In at least one embodiment, said pattern is caused by one or more of at least one interaction with an affinity reagent, at least one concentration gradient, at least one concentration gradient that causes analytes to form in a predictable spatial pattern in a spot, one or more lipid rafts and/or similar structures becoming attached to the spot, at least one variation in mutual solubility of analytes of interest and/or other chemicals driving self-sorting, or another reason.

EMBODIMENT 123: OMV and MV

Perform the method of one or more of EMBODIMENTS 101-122 above. In at least one embodiment the methods are performed on a bacterial outer membrane vesicle (OMV), a bacterial membrane vesicle (MV), or any other membrane.

EMBODIMENT 124: Environmental Response

Perform the method of one or more of EMBODIMENTS 101-123 above. In at least one embodiment, a response of at least one cell, cell culture, tissue, microbial community, biofilm, or similar sample is determined and/or measured. In at least one embodiment, a response is determined and/or measured for the purpose of determining and/or measuring antibiotic resistance and/or susceptibility. In at least one embodiment a response is determined and/or measured for the purpose of determining and/or measuring one or more of an ADME-Tox (absorption/administration, distribution, metabolism, excretion, and toxicity) parameter or other measure, efficacy, release profile, toxicity, toxicology, and/or drug treatment effect for at least one substance.

U.S. provisional patent application No. 63/054,725, filed Jul. 21, 2020, to which this application claims priority, is hereby incorporated herein by reference in its entirety. The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
using an apparatus to transfer material from a source location to a target location; and
performing a spectroscopic analysis on the material at the target location;
wherein the apparatus comprises a head portion configured to movably receive a filament having a first end for carrying the material, a transporting device configured to transport the head portion relative to the source location and relative to the target location, a driving device configured to advance the filament towards the head portion and to retract the filament away from the head portion, and a trimming device configured to trim the first end of the filament;
wherein using the apparatus includes moving the first end of the filament into contact with the material at the source location, moving the first end of the filament and a portion of the material coupled to the first end of the filament from the source location to the target location, and moving the portion of the material coupled to the first end of the filament into contact with the target location to deposit the portion of the material at the target location;
wherein using the apparatus includes, after the portion of the material is deposited at the target location, trimming the first end of the filament.

2. The method according to claim 1 wherein the filament includes a second end opposite the first end, wherein the second end of the filament is accommodated in a filament storage unit located at a fixed location relative to the head portion of the apparatus or at a fixed location relative to the driving device of the apparatus.

3. The method according to claim 1 wherein the filament includes a second end opposite the first end, wherein the second end of the filament is accommodated in a movable filament storage unit.

4. The method according to claim 1 wherein the driving device is coupled to the head portion, mounted on a fixed support separated from the head portion, or mounted on a movable support separated from the head portion.

5. The method according to claim 1 wherein the target location is not in a well plate or receptacle.

6. A method, comprising:
transferring material from a source location to a target location by positioning a first end of a filament at the source location to collect material on the first end of the filament and then positioning the first end of the filament at the target location to deposit some or all of the material at the target location; and
performing a spectroscopic analysis on the material at the target location.

7. The method of claim 6, further comprising wiping, tamping, and/or vibrating the filament to encourage release of the material at the target location and/or to spread the material at the target location.

8. The method of claim 6 wherein the target location is on a matrix-assisted laser desorption/ionization plate and the spectroscopic analysis is matrix-assisted laser desorption/ionization mass spectrometry.

9. The method of claim 6 wherein the target location is on a plate configured for use in Raman spectroscopy and the spectroscopic analysis is Raman spectroscopy.

10. The method of claim 6 wherein the material is derived from a biological sample and the material is analyzed to determine the presence, concentration, or absence of one or more bacteria, fungi, viruses, protozoans, or other parasites or organisms.

11. The method of claim 6 wherein the material is derived from urine, blood, a sample incubated in a blood bottle, sputum, endotracheal aspirate, bronchoalveolar lavage, feces, wound effluent, mucus, buccal swab, nasal swab, vaginal swab or secretion, nipple aspirate, sweat, saliva, semen or ejaculate, synovial fluid, cerebrospinal fluid, biopsy or other tissue sample, skin surface sample, tears, urinary catheter sample, culture plate, other clinical or medical sample, or another human, mammalian, or non-mammalian material.

12. The method of claim 11 wherein the sample is a clinical sample and the spectroscopic analysis is a diagnostic test.

13. The method of claim 6 wherein subsequent analysis is performed to determine one or more of microbial species ID, microbial ID at a level of specificity above or below the level of species, antimicrobial resistance, antimicrobial susceptibility, microbial growth, and/or environmental response.

14. The method of claim 13 wherein the subsequent analysis is performed entirely or predominantly on hydrophobic microbial molecules or lipids whether or not hydrophobic, including phospholipids.

15. The method of claim 13 wherein the subsequent analysis includes extracting microbial membrane lipids.

16. The method of claim 13 wherein the subsequent analysis is for identifying and/or classifying microorganisms in one or more samples.

17. The method of claim 13 wherein the subsequent analysis is for detecting and/or measuring antimicrobial resistance and/or susceptibility of a microorganism in one or more samples, and/or for estimating the minimum inhibitory concentration of a antimicrobial agent for a microorganism in one or more samples.
